(19) United States
(12) Patent Application Publication
(10) Pub. No.: US 2026/0315533 A1
(43) Pub. Date: Oct. 15, 2026

(54) IMMUNE CELL TREATMENT OF NERVE DAMAGE

(71) Applicant: OHLABBIO CO., LTD., Seoul (KR)

(72) Inventors: Seog Bae OH, Seoul (KR); Alexander James Davies, Guru-si (KR); Hyoung Woo Kim, Jeonju-si (KR); Wooin Lee, Siheung-si (KR); Kihwan Lee, Seoul (KR); Sanghyeon Lee, Bucheon-si (KR)

(73) Assignee: OHLABBIO CO., LTD., Seoul (KR)

(21) Appl. No.: 19/464,570

(22) Filed: Jan. 29, 2026

Related U.S. Application Data
(63) Continuation-in-part of application No. 17/139,618, filed on Dec. 31, 2020, now abandoned.
Application No. 17/139,618 is a continuation-in-part of application No. PCT/KR2019/008066, filed on Jul. 2, 2019.

(30) Foreign Application Priority Data
Jul. 4, 2018 (KR) 10-2018-0077922

(51) Int. Cl. A61K 35/17; A61K 9/00; A61K 38/17; A61K 38/20; A61K 40/15; A61K 40/31; A61K 45/06; A61P 25/02; C12N 5/0783

(52) U.S. Cl. CPC A61K 35/17; A61K 9/0019; A61K 38/177; A61K 38/20; A61K 40/15; A61K 40/31; A61K 45/06; A61P 25/02; C12N 5/0646; A61K 2239/21; A61K 2239/22; A61K 2239/31; A61K 2239/38; C12N 2501/2302

(57) ABSTRACT

Provided is a composition for treating nerve injury, the composition including a NK cell, an immune cell, or a substance increasing activity thereof. According to the NK cell, the immune cell, or the substance increasing activity thereof according to an aspect, the NK cell may infiltrate into a nerve injury site to directly remove injured nerves, thereby being usefully applied to fundamental treatment of a nervous system disease caused by nerve injury or abnormal nerves.

### CROSS-REFERENCE TO RELATED APPLICATIONS

This application is a Continuation-in-Part of U.S. application Ser. No. 17/139,618, filed Dec. 31, 2020, which is a Continuation-in-Part of PCT International Patent Application No. PCT/KR2019/008066, filed Jul. 2, 2019, which claims priority to Korean Patent Application No. 10-2018-0077922, filed Jul. 4, 2018, the disclosures of which are incorporated by reference herein in their entireties.

### TECHNICAL FIELD

The present disclosure relates to a composition for treating nerve injury, the composition including a natural killer cell, an immune cell, or a substance increasing activity thereof.

### BACKGROUND ART

The immune system functions to protect the body from harmful substances. Harmful substances to which the immune system responds are known to include bacteria, viruses, toxins, cancer cells, or blood and tissues from another individual. Immunotherapies have been developed to regulate the activity of the immune system in a variety of diseases where the immune system acts. However, among the therapies, a method of using an immune cell therapeutic agent is currently used only for cancer treatment.

On the other hand, neuropathy caused by nerve injury due to diabetes, anticancer drugs, trauma, etc. causes severe chronic pain and movement disorders, but symptomatic drug therapy is mainly used as a treatment method. For example, drugs known to suppress pain, such as tricyclic antidepressants, anticonvulsants, serotonin and noradrenaline reuptake inhibitors, and opioids, have been used to reduce pain caused by peripheral neuropathy. These drugs cause various side effects, and they only suppress symptoms. There is no known method of treating injured nerves, which is the underlying cause.

Accordingly, it is necessary to develop a new treatment method of using immune cells, based on the underlying cause of neuropathy, to overcome the above problems.

### DESCRIPTION OF EMBODIMENTS

### Technical Problem

An aspect provides a composition for preventing or treating a nervous system disease caused by nerve injury or abnormal nerves, the composition including an isolated natural killer cell, a cell population thereof, a substance increasing natural killer cell activity, or a combination thereof.

Another aspect provides a method of preventing or treating a nervous system disease caused by nerve injury or abnormal nerves, the method including administering to a subject in need thereof an effective amount of an isolated natural killer cell, a cell population thereof, a substance increasing natural killer cell activity, or a combination thereof.

Still another aspect provides use of an isolated natural killer cell, a cell population thereof, a substance increasing natural killer cell activity, or a combination thereof in preparing a pharmaceutical composition for preventing or treating a nervous system disease caused by nerve injury or abnormal nerves.

Still another aspect provides a method of removing injured or abnormal nerves, the method including administering to a subject in need thereof an effective amount of an isolated natural killer cell, a cell population thereof, a substance increasing natural killer cell activity, or a combination thereof.

### Solution to Problem

An aspect provides a composition for preventing or treating a nervous system disease caused by nerve injury or abnormal nerves, the composition including an isolated natural killer (NK) cell, a cell population thereof, a substance increasing natural killer cell activity, or a combination thereof.

As used herein, the term “treatment” refers to or includes alleviation, inhibition of progression, or prevention of a disease, a disorder or condition, or one or more symptoms thereof, and the “active ingredient” or “pharmaceutically effective amount” means any amount of the composition provided herein to carry out the present disclosure, which is sufficient for alleviation, inhibition of progression, or prevention of a disease, a disorder or condition, or one or more symptoms thereof.

As used herein, the terms “administering”, “introducing”, and “transplanting” are used interchangeably with each other, and refer to placement of the composition according to a specific embodiment into an individual by a method or route which results in at least partial localization of the composition according to a specific embodiment at a desired site. The cells of the composition according to a specific embodiment or at least a portion thereof may be administered by any appropriate route that results in delivery to a desired site in a living individual. A period of viability of the cells after being administered to the individual may be as short as a few hours, e.g. twenty four hours, to a few days, to as long as several years.

As an aspect, the composition including the isolated NK cell, the cell population thereof, is administered to a human subject, wherein the NK cells are human-derived NK cells.

As another aspect, for non-clinical evaluation and validation of the therapeutic concept, the composition including the animal-derived NK cell (e.g. mouse-derived NK cell), the cell population thereof, may be treated to a corresponding animal (e.g. mouse) disease model.

As used herein, the term “isolated cell”, e.g., “isolated natural killer cell”, etc. refers to a cell substantially isolated from a tissue from which the cell is originated, e.g., hematopoietic cells.

As used herein, the term “immune cell” refers to a cell that regulates immunity against pathogens or toxins that invades a living organism, and may include natural killer cell, T cell, T lymphocyte, B cell, dendritic cell, or macrophage. Further, the immune cell may be an autologous immune cell or an allogeneic immune cell.

Hereinafter, the natural killer cell of the present disclosure will be described in detail.

As used herein, the term “natural killer cell” or “NK cell” is a cytotoxic lymphocyte that constitutes a major component of the innate immune system, and is defined as a large granular lymphocyte (LGL), and constitutes the third kind of cell differentiated from the common lymphoid progenitor generating B and T lymphocytes. The “natural killer cell” or “NK cell” includes a natural killer cell derived from any tissue source without additional modifications, and may include a natural killer progenitor cell as well as a mature natural killer cell. The natural killer cell is activated in response to interferon or macrophage-derived cytokine, and the natural killer cell includes two types of cell surface receptors that control cytotoxic activity of the cell, labeled as “activating receptors” and “inhibiting receptors”. The natural killer cell may be generated from hematopoietic cells, e.g., hematopoietic stem or progenitor cells, from any source, e.g., a placental tissue, a placental perfusate, umbilical cord blood, placental blood, peripheral blood, spleen, liver, etc.

In a specific embodiment, the natural killer cell may be an activated natural killer cell. The activated natural killer cell may be a cell in which cytotoxicity or intrinsic immunomodulatory ability of the natural killer cell is activated, as compared with a mother cell, e.g., a hematopoietic cell or a natural killer progenitor cell. In a specific embodiment, the activated natural killer cells are CD3⁻CD56⁺. In a specific embodiment, the activated natural killer cells are CD3⁻CD56⁺CD16⁻. In another specific embodiment, the activated natural killer cells are additionally CD94⁺CD117⁺. In another specific embodiment, the activated natural killer cells are additionally CD161⁻. In another specific embodiment, the activated natural killer cells are additionally NKG2D⁺. In another specific embodiment, the activated natural killer cells are additionally NKp46⁺. In another specific embodiment, the activated natural killer cells are additionally CD226⁺. In a specific embodiment, the activated natural killer cells are additionally granzyme B⁺. In a specific embodiment, the activated natural killer cells are additionally perforin⁺. In a specific embodiment, more than 50%, 60%, 70%, 80%, 90%, 92%, 94%, 96%, or 98% of the activated natural killer cells are CD56⁺ and CD16⁻. In another specific embodiment, at least 50%, 60%, 70%, 80%, 82%, 84%, 86%, 88%, or 90% of the activated natural killer cells are CD3⁻ and CD56⁺. In another specific embodiment, at least 50%, 52%, 54%, 56%, 58%, 60%, 70%, 80%, 90%, 92%, 94%, 95%, 96%, 97%, 98%, or 99% of the activated natural killer cells are NKG2D⁺. In another specific embodiment, at least 50%, 52%, 54%, 56%, 58%, 60%, 70%, 80%, 90%, 92%, 94%, 95%, 96%, 97%, 98%, or 99% of the activated natural killer cells are granzyme B⁺. In another specific embodiment, at least 50%, 52%, 54%, 56%, 58%, 60%, 70%, 80%, 90%, 92%, 94%, 95%, 96%, 97%, 98%, or 99% of the activated natural killer cells are perforin⁺. In another specific embodiment, 30%, 20%, 10%, 9%, 8%, 7%, 6%, 5%, 4%, or 3% of the cells are NKB1⁺. In a specific embodiment, less than 30%, 20%, 10%, 8%, 6%, 4%, or 2% of the activated natural killer cells are NKAT2⁺. In another specific embodiment, less than 30%, 20%, 10%, 8%, 6%, 4%, or 2% of the activated natural killer cells are CD56⁺ and CD16⁺. In a more specific embodiment, at least 10%, 20%, 25%, 30%, 35%, 40%, 50%, 55%, 60%, 65%, or 70% of the CD3⁻ and CD56⁺ activated natural killer cells are NKp46⁺. In another more specific embodiment, at least 10%, 20%, 25%, 30%, 35%, 40%, 50%, 55%, 60%, 65%, 70%, 75%, 80%, or 85% of the CD3⁻ and CD56⁺ activated natural killer cells are CD117+. In another more specific embodiment, at least 10%, 20%, 25%, 30%, 35%, 40%, 45%, or 50% of the CD3⁻ and CD56⁺ activated natural killer cells are CD94⁺. In another more specific embodiment, at least 10%, 20%, 25%, 30%, 35%, 40%, 45%, or 50% of the CD3⁻ and CD56⁺ activated natural killer cells are CD161⁻. In another more specific embodiment, at least 10%, 12%, 14%, 16%, 18%, or 20% of the CD3⁻ and CD56⁺ activated natural killer cells are CD226⁺. In another more specific embodiment, at least 20%, 25%, 30%, 35%, or 40% of the CD3⁻ and CD56⁺ activated natural killer cells are CD7⁺. In another more specific embodiment, at least 30%, 35%, 40%, 45%, 50%, 55%, or 60% of the CD3⁻ and CD56⁺ activated natural killer cells are CD5⁺. In another more specific embodiment, at least 30%, 35%, 40%, 45%, 50%, 55%, 60%, 65%, 70%, 75%, 80%, 85%, 90%, 92%, 94%, or 95% of the CD3⁻ and CD56⁺ activated natural killer cells are perforin⁺. In another more specific embodiment, at least 30%, 35%, 40%, 45%, 50%, 55%, 60%, 65%, 70%, 75%, 80%, 85%, 90%, 92%, 94%, or 96% of the CD3⁻ and CD56⁺ activated natural killer cells are granzyme B⁺.

In a specific embodiment, the activated natural killer cell or activated natural killer cell-enriched population may be assessed by detecting one or more functionally relevant markers, e.g., CD94, CD161, NKp44, DNAM-1, 2B4, NKp46, CD94, KIR, and NKG2 family of activating receptor (e.g., NKG2D).

In a specific embodiment, the activated natural killer cell may be generated from the above-described hematopoietic cell. In a specific embodiment, the activated natural killer cell may be obtained from a proliferated hematopoietic cell, e.g., a hematopoietic stem cell and/or a hematopoietic progenitor cell. In a specific embodiment, the hematopoietic cell is proliferated and differentiated, continuously, in a first medium without the use of feeder cells. The cell is then cultured in a second medium in the presence of feeder cells. Such separation (isolation), proliferation, and differentiation may be performed in a central facility, which provides expanded hematopoietic cells for proliferation and differentiation at points of use, e.g., hospital, etc.

In a specific embodiment, the generation of the activated natural killer cell includes proliferating a population of hematopoietic cells. A plurality of hematopoietic cells within the population of hematopoietic cells differentiates into natural killer cells during the cell proliferation.

In the present disclosure, the activated natural killer cell may be produced by a two-step method of proliferation/differentiation and maturation of natural killer cell. The first and second steps include culturing the cells in a medium with a unique combination of cellular factors. In a specific embodiment, the method includes (a) culturing and proliferating a population of hematopoietic cells in a first medium containing interleukin, etc., wherein a plurality of hematopoietic stem or progenitor cells within the hematopoietic cell population are differentiated into natural killer cells; and (b) proliferating the natural killer cells from (a) in a second medium containing interleukin, etc., wherein the natural killer cells are further proliferated and differentiated, and the natural killer cells are maturated (e.g., activated or otherwise possessing cytotoxic activity). In another specific embodiment, the method includes no intermediary process between (a) and (b), no additional culturing process prior to (a), and/or no additional process (e.g., maturation) after (b).

As used herein, the term “natural killer progenitor cell”, or “NK progenitor cell”, or cell population thereof may refer to, for example, a cell including a natural killer cell lineage that has not yet developed into mature natural killer cells, or a population thereof, as represented by expression levels of one or more phenotypic markers, e.g., CD56, CD16, and KIR. In one embodiment, the natural killer progenitor cell population includes cells having low CD16 and high CD56. For example, the natural killer progenitor cell population includes about 5%, about 10%, about 15%, about 20%, about 25%, about 30%, about 35%, about 40%, about 45%, or about 50% of CD3-CD56⁺ cells. In another specific embodiment, the natural killer progenitor cell population includes 5%, 10%, 15%, 20%, 25%, 30%, 35%, 40%, 45%, or 50% or less of CD3⁻CD56⁺ cells. In another specific embodiment, the natural killer progenitor cell population includes 0%-5%, 5%-10%, 10%-15%, 15%-20%, 20%-25%, 25%-30%, 30%-35%, 35%-40%, 40%-45%, or 45%-50% of CD3⁻CD56⁺ cells.

In a specific embodiment, the CD3⁻CD56⁺ cells in the natural killer progenitor cell population are additionally CD117⁺. In a specific embodiment, about 65%, about 70%, about 75%, about 80%, about 85%, about 90%, about 95%, about 98%, or about 99% of the CD3⁻CD56⁺ cells in the natural killer progenitor cell population are CD117⁺. In another specific embodiment, 65%, 70%, 75%, 80%, 85%, 90%, 95%, 98%, or 99% or more of the CD3⁻CD56⁺ cells in the natural killer progenitor cell population are CD117⁺. In another specific embodiment, 65%-70%, 70%-75%, 75%-80%, 80%-85%, 85%-90%, 90%-95%, or 95%-99% of the CD3⁻CD56⁺ cells in the natural killer progenitor cell population are CD117⁺.

In another specific embodiment, the CD3⁻CD56⁺ cells in the natural killer progenitor cell population are additionally CD161⁺. In a specific embodiment, about 40%, about 45%, about 50%, about 55%, about 60%, about 65%, about 70%, or about 75% of the CD3⁻CD56⁺ cells in the natural killer progenitor cell population are CD161⁺. In another specific embodiment, 40%, 45%, 50%, 55%, 60%, 65%, 70%, or 75% or more of the CD3⁻CD56⁺ cells in the natural killer progenitor cell population are CD161⁺. In another specific embodiment, 40%-45%, 45%-50%, 50%-55%, 55%-60%, 60%-65%, 65%-70%, or 70%-75% of the CD3⁻CD56⁺ cells in the natural killer progenitor cell population are CD161⁺.

In still another specific embodiment, the CD3⁻CD56⁺ cells in the natural killer progenitor cell population are additionally NKp46⁺. In a specific embodiment, about 25%, about 30%, about 35%, about 40%, about 45%, about 50%, about 55%, about 60%, about 65%, about 70%, about 75%, about 80%, about 85%, about 90% or more of the CD3⁻CD56⁺ cells in the natural killer progenitor cell population are NKp46⁺. In another specific embodiment, about 25%, about 30%, about 35%, about 40%, about 45%, about 50%, or about 55% of the CD3⁻CD56⁺ cells in the natural killer progenitor cell population are NKp46⁺. In another specific embodiment, 25%, 30%, 35%, 40%, 45%, 50%, or 55% or less of the CD3⁻CD56⁺ cells in the natural killer progenitor cell population are NKp46⁺. In another specific embodiment, 25%-30%, 30%-35%, 35%-40%, 40%-45%, 45%-50%, 50%-55%, 55%-60%, 60%-65%, 65%-70%, 70%-75%, 75%-80%, 80%-85%, 85%-90% or more of the CD3⁻CD56⁺ cells in the natural killer progenitor cell population are NKp46⁺. In another more specific embodiment, 25%-30%, 30%-35%, 35%-40%, 40%-45%, 45%-50%, or 50%-55% of the CD3⁻CD56⁺ cells in the natural killer progenitor cell population are NKp46⁺.

Further, the natural killer progenitor cell population is the same as for CD52⁺, CD16⁺, CD244⁺ CD94⁺, or CD94⁺.

In the present disclosure, the natural killer cell may be genetically modified or engineered.

As used herein, “genetic modification” or “genetic engineering” includes artificially altering a composition or structure of the cell's genetic material.

In a specific embodiment, the natural killer cell may be genetically modified to have improved target specificity and/or homing specificity.

In a specific embodiment, the genetically modified natural killer cell is a natural killer cell including chimeric antigen receptor (CAR). CAR is an artificial membrane-bound protein that directs an immune cell (e.g., T lymphocyte) to an antigen, and stimulates the immune cell to kill a cell displaying the antigen. CAR includes an extracellular domain that binds to an antigen, e.g., an antigen on a cell, a transmembrane domain, and an intracellular (cytoplasmic) signaling domain (i.e., intracellular stimulatory domain) that transmits a primary activation signal to an immune cell, and/or a co-stimulating domain. All other conditions being satisfied, when CAR is expressed on the surface of, e.g., T lymphocyte, for example, primary T lymphocyte, and the extracellular domain of CAR binds to an antigen, the extracellular signaling domain transmits a signal to the T lymphocyte to activate and/or proliferate, and if the antigen is present on a cell surface, to kill the cell expressing the antigen. Some immune cells, e.g., T lymphocytes and natural killer cells, require two signals, i.e., a primary activation signal and a costimulatory signal, in order to maximally activate, and CARs also include a costimulatory domain such that binding of the antigen to the extracellular domain results in transmission of both the primary activation signal and the costimulatory signal.

In another specific embodiment, the genetically modified natural killer cell may be a natural killer cell including a homing receptor. This causes the cell including a homing receptor to home to a particular anatomical zone, particularly, a tissue, or a particular type of cell, e.g., B cell zone of lymph nodes, gastrointestinal tract, or skin.

In still another specific embodiment, the genetically modified natural killer cells are natural killer cells including both CAR and homing receptor as described herein.

The natural killer cells including CAR and/or homing receptor may be generated by any method known in the art. In some embodiments, the natural killer cells including CAR and/or homing receptor are engineered to express CAR and/or homing receptor by introducing (e.g., by transfection), into the natural killer cells, one or more vectors including a nucleotide sequence(s) encoding CAR and/or homing receptor. In some embodiments, the cells (e.g., CD34⁺ hematopoietic stem cells), from whom natural killer cells may be produced, are first engineered to express CAR and/or homing receptor by introducing (e.g., by transfection), into the cells, one or more vectors including the nucleotide sequence(s) encoding CAR and/or homing receptor, and the cells are then used to derive natural killer cells including CAR and/or homing receptor by the method described above.

In a specific embodiment, the extracellular domain of CAR is an antigen binding domain. In a specific embodiment, the antigen binding domain is a scFv domain. In a specific embodiment, the antigen binding domain specifically binds to TAA. In a specific embodiment, TAA is selected from the group consisting of CD123, CLL-1, CD38, CD20, and CS-1. In a more specific embodiment, the antigen-binding domain includes a single-chain Fv (scFv) or antigen-binding fragment derived from an antibody that binds to CS-1. In a more specific embodiment, the antigen-binding domain includes a single-chain of elotuzumab and/or an antigen-binding fragment of elotuzumab. In a specific embodiment, the antigen-binding domain includes a single-chain Fv (scFv) or antigen-binding fragment derived from an antibody that binds to CD20.

In a specific embodiment, the intracellular stimulatory domain of CAR is a CD3 zeta signaling domain.

In a specific embodiment, the co-stimulatory domain of CAR includes the intracellular domain of CD28, 4-1BB, PD-1, OX40, CTLA-4, NKp46, NKp44, NKp30, DAP10, or DAP12.

In a specific embodiment, the homing receptor is a chemotactic receptor. In a specific embodiment, the chemotactic receptor is selected from the group consisting of CXCR4, VEGFR2, and CCR7.

Hereinafter, the substance increasing natural killer cell activity of the present disclosure will be described in detail.

As used herein, the term “substance increasing natural killer cell activity” may refer to any substance which may be administered in vivo to increase the activity of the natural killer cell, for example, the cytotoxic activity thereof, or to generate, increase, or proliferate the activated natural killer cell.

In a specific embodiment, it was confirmed that the substance increasing natural killer cell activity as well as the natural killer cell itself was administered in vitro or in vivo to remove the injured nerves. Thus, the substance increasing natural killer cell activity according to a specific embodiment may be usefully applied in removing or treating injured nerves or abnormal nerves, or to neuropathy.

In a specific embodiment, the substance increasing natural killer cell activity may include immune cytokines, agonists of NK cell activating receptors, or antagonists of NK cell inhibitory receptors.

As used herein, the term “cytokine” may refer to a physiological regulator that is a signal substance controlling the defense system in the body and stimulating the living body. Cytokines regulate various biological activities, such as cell activation, differentiation, cell migration, aging, death induction, etc., in various cells through autocrine and paracrine actions. The cytokine may be, for example, interferon (INF) family, interleukin (IL) family, tumor necrosis factors (TNF) family, or a combination thereof. More particularly, the cytokine may be any one selected from the group consisting of IL-2, IL-5, IL-8, IL-12, IL-15, IL-18, IL-21, and a combination thereof. More particularly, the interferon may be type 1 interferon (e.g., interferon-α, interferon-β, interferon-κ, or interferon-ω), type 2 interferon (e.g., interferon-γ), type 3 interferon, or a combination thereof. Further, TNF may include TNF-α, TNF-β, TNF-γ, etc. Further, the immune cytokine may include a cytokine-antibody complex. For example, the immune cytokine may include an interleukin (e.g., IL-2)-anti-interleukin (e.g., anti-IL-2) antibody complex.

The agonists of NK cell activating receptors may include an antibody or aptamer binding to activating receptors, Bispecific Killer Engagers (BiKEs) or Trispecific Killer Engagers (TrikEs).

Examples of the antibody or aptamer binding to activating receptors may include an antibody against GITR, an antibody against OX40, an antibody against CD137, an antibody against CD27, OX40 agonistic aptamer, CD137 agonistic aptamer, NKG2D ligand (e.g., murine UL16-binding protein-like-1 (murine MULT-1) or H60, or human MHC I-related chain A (MICA), MHC I-related chain B (MICB) or unique long 16-binding proteins (ULBPs)), or CD226 agonist.

The BiKEs or TriKEs are reagents that contain two single-chain variable fragments (scFv) and specifically engage both target cells (e.g., injured nerve cells, tumor cells, or infected cells) and natural killer cells to mediate clearance of damaged nerves and killing of tumor cells or infected cells (TrikEs bind to two surface antigens or receptors of natural killer cells).

They are used to colocalize target cells (e.g., injured nerve cells, tumor cells, or infected cells) with natural killer cells, thereby triggering natural killer-cell mediated antibody-dependent cellular cytotoxicity (ADCC). BiKEs may be generated by any method known in the art, for example, as described in Gleanson, M. K., et al., Mol. Cancer Ther., 2012, 11:2674-2684; Vallera, D. A., et al., Cancer Biother. Radiopharm., 2013, 28:274-282; Wiernik, A., et al., Clin. Cancer Res., 2013, 19:3844-3855; Reiners, K. S., et al., Mol. Ther., 2013, 21:895-903; Singer, H., et al., J. Immunother., 2010, 33:599-608; or Gleason, M. K., et al., Blood, 2014, 123:3016-3026. One scFv of BIKE specifically binds to an antigen on the surface of target cells (e.g., injured nerve cells, tumor cells, or infected cells), and the other scFv specifically binds to a receptor (e.g., an Fc receptor, such as CD16) on natural killer cells. In a specific embodiment, the BiKE includes a first scFv that specifically binds to TAA or Retinoic Acid Early 1 (RAE1) and a second scFv that specifically binds to activating receptors, e.g., CD16.

The antagonists of NK cell inhibitory receptors may include an antibody or aptamer binding to inhibitory receptors. Examples of the NK cell inhibitory receptor may include CTLA-4, PD-1, PD-L1, Tim-3, CD96, KIR, NKG2A, or T-cell immunoglobulin and ITIM domain (TIGIT). Therefore, the NK cell inhibitory receptor may include antibodies against the above-described receptors.

Hereinafter, the nervous system disease caused by nerve injury or abnormal nerves of the present disclosure will be described in detail.

As used herein, the term “nerve injury” may refer to injury to nerve tissues or nerve cells, including axon degeneration, Wallerian degeneration, etc. Further, the nerve injury may include, depending on the degree of injury (in peripheral), nerve compression without Waller degeneration (neuropraxia), axonal injury with Waller degeneration (axonotmesis), or neurotmesis with loss of axonal continuity.

As used herein, the “abnormal nerve” may refer to occurrence of abnormal neuronal activity due to changes in part or all of the nerve tissues or nerve cells, caused by a congenital or acquired cause. The abnormal nerve may include changes induced by nerve injury or other factors, and may include structural or functional modifications such as degeneration, necrosis, hypertrophy, abnormal proliferation, and reduced conductive function. For example, when neurodegeneration occurs due to lack of oxygen supply caused by a blockage of blood vessels such as vascular dementia, cerebral infarction, etc., excitotoxicity in the central nervous system may be induced to cause neurodegeneration.

As used herein, the term “nervous system disease caused by nerve injury or abnormal nerves” may be a nervous system disease caused by nerve injury or abnormal nerves of the central nervous system or a nervous system disease caused by nerve injury or abnormal nerves of the peripheral nervous system, and may or may not be accompanied by symptoms such as paralysis, convulsion, seizure, cognitive disorder, speech disorder, memory disorder, behavioral abnormalities, emotional control disorder, dizziness, vomiting, gait disturbance, hormonal abnormality, dysequilibrium, pain, paresthesia, reduced motor function, numbness, tingling, burning, etc., depending on where and how the injury has occurred. For example, the nervous system disease caused by nerve injury or abnormal nerves of the central nervous system may be accompanied by main symptoms such as paralysis, convulsion, seizure, cognitive disorder, speech disorder, memory disorder, behavioral abnormalities, emotional control disorder, dizziness, vomiting, gait disturbance, hormonal abnormality, disequilibrium, etc., and the nervous system disease caused by nerve injury or abnormal nerves of the peripheral nervous system may be accompanied by main symptoms such as pain, paresthesia, reduced motor function, numbness, tingling, burning, etc. The central nervous system is a nervous system including the brain and the spinal cord. When the spinal cord is injured by trauma, diseases, etc., nerve transmission between the brain and the body may not be normally performed, resulting in paralysis of motion or sensation. The peripheral nervous system is a nervous system that contains 12 pairs of cranial nerves, 31 pairs of spinal nerves, and autonomic nerves. Depending on the site of injury, symptoms such as facial paralysis due to facial nerve injury, blindness due to optic nerve injury, eyelid paralysis due to oculomotor nerve injury, and hearing loss or dizziness due to auditory nerve injury may occur.

In a specific embodiment, the nervous system disease caused by nerve injury or abnormal nerves of the central nervous system may be any one selected from the group consisting of organic diseases and dysfunctions of the central nervous system, epilepsy, multiple sclerosis, amyotrophic lateral sclerosis, Alzheimer's disease, Lewy dementia, Huntington's disease, Parkinson's disease, schizophrenia, traumatic brain injury, stroke, Pick's disease, Creutzfeldt-Jakob disease, progressive supernuclear palsy, multiple system atrophy, corticobasal degeneration, spinocerebellar degeneration, cerebellar atrophy, post-traumatic stress disorder, amnesia, vascular dementia, and cerebral infarction. As used herein, the “organic diseases and dysfunctions of the central nervous system” may refer to a disease showing symptoms, such as paralysis, convulsion, seizure, cognitive disorder, speech disorder, memory disorder, behavioral abnormalities, emotional control disorder, dizziness, vomiting, gait disturbance, hormonal abnormality, dysequilibrium, pain, etc., due to nerve injury or abnormal nerve of the central nervous system. In a specific embodiment, the nervous system disease caused by nerve injury or abnormal nerves of the central nervous system may be associated with excitotoxicity in the central nervous system. For example, the disease may be epilepsy, multiple sclerosis, amyotrophic lateral sclerosis, Alzheimer's disease, Lewy dementia, Huntington's disease, Parkinson's disease, schizophrenia, traumatic brain injury, stroke, etc., and may also include degenerative brain diseases associated with excitotoxicity in the central nervous system.

Epilepsy can be induced by traumatic injuries, such as blast-related trauma, penetrating brain injuries, and ischemic brain injuries (Exp. Neurol., 2009, 215(2): 243-252; J. Neurotrauma. 2011, 28:71-83; J. Neurotrauma., 2013, 30:1973-1982; J. Neurotrauma., 2013, 30:580-590.; Neurotherapeutics, 2014, 11:286-296).

Multiple sclerosis (MS) has been reported to be associated with an increased risk after head trauma (J. Neurol. Neurosurg. Psychiatry, 2024, 95:554-560), and the expression of NKG2D ligand (ULBP4) is upregulated in the central nervus system (CNS) and cerebrospinal fluid (CSF) of patients with the multiple sclerosis (Neurol. Neuroimmunol. Neuroinflamm., 2022, 9:e1119).

Amyotrophic lateral sclerosis (ALS) can be aggravated by nerve injury, which induces motor neuron loss and inflammation (J. Neuroinflammation, 2020, 17:175). NKG2D ligand expression has been reported in motor neurons in mouse models of ALS and human patients (Nat. Commun., 2020, 11:1773).

Alzheimer's disease can be aggravated, and its progression can be accelerated by traumatic brain injuries via chronic neuroinflammation and reactivation of latent neurotropic viruses (e.g. HSV-1) (J. Neurol. Neurosurg. Psychiatry, 2003, 74:857-862; Neurosurgery, 2005, 57:719-726; Sci. Signal., 2025, 18:eado6430). It has been reported that neurodegenerative stress or amyloid-beta accumulation can upregulate NKG2D ligands on vulnerable neurons in Alzheimer's disease (Neurochem. Int., 2025, 184:105947).

The levels of NKG2D ligands, such as MICA and MICB, are reported to be upregulated in Parkinson's disease, which can induce interaction with NK cells within the CNS (Exp. Mol. Med., 2020, 52:1517-1525).

After stroke, the expression of NKG2D ligands, such as ULBP1 (MULT1) in mice and ULBP-1, -3, -4 in human, is significantly increased in murine brain and in the brain tissues of human patients (J Am Heart Assoc., 2023, 12:e029529).

Tauopathies are a group of diseases featuring progressive dying-back neurodegeneration of specific neuronal populations in association with accumulation of abnormal forms of the microtubule-associated protein tau and comprise a heterogeneous group of diseases, including Alzheimer's disease (AD), frontal temporal dementia with parkinsonism linked to chromosome17 (FTDP-17), chronic traumatic encephalopathy (CTE), progressive supranuclear palsy (PSP), corticobasal degeneration (CBD), and Pick's disease (PiD). In human tauopathies and animal models, axonal pathology and degeneration have been determined (Front. Neurosci., 2017, 11:572).

In progressive supranuclear palsy (PSP), plasma neurofilament light chain (NfL) concentrations are elevated and twice as high as those in controls, which indicates axonal damages in PSP patients (Ann. Clin. Transl. Neurol., 2016; 3(3):216-225).

In patients with Multiple system atrophy (MSA), plasma NfL levels have been reported to be higher than in healthy controls, indicating axonal injury in MSA (Neurology, 2002, 98:e73-e82).

Spinocerebellar ataxia, a subtype of spinocerebellar degeneration, is associated with peripheral nerve involvement, which manifests not only as distal axonal neuropathy but also as primary neuronopathy (Arch. Neurol., 2004, 61:257-261).

Cerebellar atrophy can be aggravated by traumatic head (brain) injuries (Child's Nerv. Syst., 2001, 17:263-269; AJNR Am. J. Neuroradiol., 2007, 28(3):537-542).

Post-traumatic stress disorder (PTSD) and traumatic brain injury show significantly high comorbidity and share symptomology, which indicates the significant overlap in underlying pathophysiology. Both neurological disorders are related to neuroinflammation, oxidative stress, excitotoxicity, and structural changes. The traumatic brain injury can exacerbate PTSD via neuroinflammation (Exp. Neurol., 2022, 351:114009).

Amnesia can be caused by traumatic brain injury to the major structures of the diencephalon (Brain Injury, 2009, 23(1):71-76; Brain Injury, 2000, 14(9):765-780).

Vascular dementia (VaD) arises from complications of cerebrovascular disease and is associated with vascular injury-induced neuronal damage. Neurofilament light chain (NfL) levels may serve as a fluid-based biomarker for VaD (J. Clin. Med., 2025, 14:6611).

In a specific embodiment, the nervous system disease caused by nerve injury or abnormal nerves of the peripheral nervous system may be any one selected from the group consisting of peripheral neuropathy, diabetic neuropathy, peripheral neuropathic pain, chemotherapy-induced peripheral neuropathy, complex regional pain syndrome (CRPS), optic neuropathy, mononeuropathy, mononeuropathy multiplex (mononeuritis multiplex), polyneuropathy, Guillain-Barre syndrome (acute inflammatory demyelinating polyneuropathy), chronic inflammatory demyelinating polyneuropathy, hereditary neuropathy, plexus disorder, glaucoma, macular degeneration, amyotrophic lateral sclerosis, progressive muscle atrophy, progressive bulbar palsy, polio, post-polio syndrome, stiff-man syndrome, Isaac's syndrome, myasthenia gravis, neonatal myasthenia, botulism, Lambert-Eaton Myasthenic Syndrome (LEMS), thoracic outlet syndrome, Charcot-Marie-Tooth disease, fibromyalgia syndrome (FMS), and spinal muscular atrophy.

Peripheral neuropathy (or peripheral neuropathic pain) can induce NKG2D ligands, such as RAE1, expression in adult dorsal root ganglion neurons, thereby promoting interaction with NK cells (Cell, 2019, 176:716-728; Front. Neurosci., 2020, 14:142; Trends in Neurosciences, 2023, 46(8): p617-627).

The pathogenesis of diabetic neuropathy is associated with abnormal metabolic factors, disrupted peripheral nervous system, including myelinated and unmyelinated axons, neuroinflammation, and oxidative stress (Front. Endocrinol., 2024, 14:1265372).

Chemotherapy-induced peripheral neuropathy (CIPN) is one of the most frequent side effects caused by antineoplastic agents, and CIPN occurs due to the neurotoxicity of the antineoplastic agents, which damage nervous system structures (e.g. demyelination) (Int. J. Mol. Sci., 2019, 20:1451).

Optic neuropathy or traumatic optic neuropathy can occur as a result of optic nerve injury following head or facial trauma, and the mechanisms of injury may include direct or indirect damage caused by external impact, as well as secondary injury resulting from hemorrhage, ischemia, or edema after the initial insult (J. Neurol. Surg. B, 2021, 82:107-115).

Charcot-Marie-Tooth disease (CMT) can be caused by demyelination or dysmyelination of axons (Neural Regen. Res., 2023, 18(9):1931-1939).

Thoracic Outlet Syndrome (TOS) can be caused by nerve compression, which induces nerve injury and chronic axonal loss (International Orthopaedics, 2025, 49:827-834; The American Surgeon™ 2025, 91(12):2164-2172).

Poliovirus primarily damages lower motor neuron cell bodies, leading to secondary degeneration of motor axons, and in post-polio syndrome, progressive loss of previously reinnervated axons accompanied by ongoing cycles of denervation and reinnervation results in chronic neurodegeneration and low-grade inflammation (Arch. Neur. Psych., 1933, 29(2):344-358; Ann. N. Y. Acad. Sci., 1995, 753:167-85).

Complex regional pain syndrome (CRPS) can be subdivided into type I and type II, and CRPS II occurs after damage to a peripheral nerve (Autoimmunity Reviews, 2014, 13:242-265).

Skin biopsies from patients with fibromyalgia syndrome (FMS) have demonstrated increased expression of an NKG2D ligand, ULBP, in the dermal nerve fibers (Pain, 2022, 163:e821-e836).

In a specific embodiment, it has been determined that NKG2D ligand expression can be increased following traumatic injuries and/or axonal injuries and activated NK cells are cytotoxic to NKG2D ligand-expressing nerves.

In a specific embodiment, the diabetic neuropathy may be polyneuropathy or focal neuropathy. In a specific embodiment, the polyneuropathy may be any one selected from the group consisting of hyperglycemic neuropathy, distal symmetric polyneuropathy, autonomic neuropathy, acute sensory neuropathy, acute painful sensory neuropathy, and chronic sensorimotor neuropathy. In a specific embodiment, the focal neuropathy may be any one selected from the group consisting of cranial neuropathy, truncal neuropathy, limb neuropathy, thoracolumbar radiculoneuropathy, and lumbosacral radiculoplexus neuropathy.

As used herein, the term “neuropathic pain” may be a pain initiated or caused by a primary lesion or dysfunction of the nervous system, defined by IASP (Classification of chronic pain, 2nd Edition, IASP Press, 2002, 210). Neuropathic pain is a representative type of chronic, non-malignant pain, resulting from injury or malfunction of the peripheral or central nervous system, and does not provide a biological function of protection. Etiologically, neuropathic pain may be caused by trauma, surgery, spinal disc herniation, spinal cord injury, diabetes, herpes zoster infection, HIV/AIDS, terminal cancer, surgical amputation including mastectomy, carpal tunnel syndrome, chronic alcohol intake, unexpected side effects of neurotoxic agents such as radiation exposure, specific anti-HIV and chemotherapy drugs.

In the present disclosure, the “neuropathic pain induced by nerve injury or abnormal nerve of the peripheral nervous system” or “peripheral neuropathic pain” may refer to neuropathic pain initiated or induced by primary injury or dysfunction of the peripheral nervous system.

In a specific embodiment, the peripheral neuropathic pain may include pain caused by dysfunction or injury of the peripheral nervous system, pain caused by trigeminal neuralgia, diabetic neuropathic pain, phantom limb pain, viral infection, trauma, cancer, or alcoholism, pain following chemotherapy, atypical facial pain, post herpetic neuralgia, and neuropathic pain caused by neurological disorder.

In a specific embodiment, the diabetic neuropathy may be polyneuropathy or focal neuropathy. In a specific embodiment, the polyneuropathy may be any one selected from the group consisting of hyperglycemic neuropathy, distal symmetric polyneuropathy, autonomic neuropathy, acute sensory neuropathy, acute painful sensory neuropathy, and chronic sensorimotor neuropathy. In a specific embodiment, the focal neuropathy may be any one selected from the group consisting of cranial neuropathy, truncal neuropathy, limb neuropathy, thoracolumbar radiculoneuropathy, and lumbosacral radiculoplexus neuropathy.

The isolated natural killer cell, the immune cell, the cell population thereof, the substance increasing activity of natural killer cell or immune cell, or a combination thereof may promote the removal of injured or abnormal axons (nerves). Therefore, in addition to the above diseases, symptoms such as paralysis, convulsion, seizure, cognitive disorder, speech disorder, memory disorder, behavioral abnormalities, emotional control disorder, dizziness, vomiting, gait disturbance, hormonal abnormality, dysequilibrium, pain, paresthesia, reduced motor function, numbness, tingling, burning, etc., which is caused by injury or abnormality may be alleviated by removing the injured or abnormal nerves, and in particular, they may be usefully applied in alleviating the symptoms by surgically removing a problematic site or inhibiting nerve activity.

In a specific embodiment, the natural killer cell, the immune cell or the cell population thereof, the genetically modified natural killer cell, the immune cell or the cell population thereof may be used, e.g., administered to an individual, in any amount or number that results in a detectable therapeutic benefit to the individual, e.g., an effective amount. The cells may be administered to such an individual by absolute or relative numbers of cells, e.g., the individual may be administered at about, at least about, or at most about 1×10⁵ cells, about 5×10⁵ cells, about 1×10⁶ cells, about 5×10⁶ cells, about 1×10⁷ cells, about 5×10⁷ cells, about 1×10⁸ cells, about 5×10⁸ cells, about 1×10⁹ cells, about 5×10⁹ cells, about 1×10¹⁰ cells, about 5×10¹⁰ cells, or about 1×10¹¹ cells. Further, the substance increasing natural killer cell activity according to a specific embodiment may be included in an amount of 0.001% by weight to 80% by weight with respect to the total weight of the composition. An administration dosage of the substance increasing natural killer cell activity may be 0.01 mg to 10,000 mg, 0.1 mg to 1000 mg, 1 mg to 100 mg, 0.01 mg to 1000 mg, 0.01 mg to 100 mg, 0.01 mg to 10 mg, or 0.01 mg to 1 mg. However, the administration dosage may be variously prescribed in consideration of factors such as a formulation method, an administration mode, a patient's age, body weight, sex, and pathological conditions, food, administration time, administration route, excretion rate, and responsiveness. These factors may be taken into consideration to appropriately adjust the administration dosage by one of ordinary skill in the art. The dosage may be administered once or twice or more within the scope of clinically acceptable side effects, and the administration site may be one or two or more. For animals other than humans, the same dosage as used for the human per kg, or the amount of the dose converted in terms of the volume ratio (e.g., average value) of the organ of the target animal to the human organ (heart, etc.) may be used for the administration. Available routes of administration may include oral, sublingual, parenteral (e.g. subcutaneous, intramuscular, intraarterial, intraperitoneal, intradural, or intravenous), rectal, and topical routes (including transdermal), inhalation, and injection, or insertion of a portable device or substance. More specifically, administering of the population of isolated natural killer cell or the pharmaceutical composition thereof to an individual may be performed by injection, infusion, intravenous administration, intrafemoral administration, or administration into a pain site. An animal to be treated according to an embodiment includes humans and other target mammals, specifically, humans, monkeys, mice, rats, rabbits, sheep, cows, dogs, horses, pigs, etc.

The pharmaceutical composition according to an embodiment may include a pharmaceutically acceptable carrier and/or additive. For example, sterilized water, physiological saline, buffers for common use (phosphoric acid, citric acid, other organic acids, etc.), stabilizers, salts, antioxidants (ascorbic acid, etc.), surfactants, suspending agents, isotonic agents, preservatives, etc. may be included. For topical administration, organic materials such as biopolymers, inorganic materials such as hydroxyapatite, specifically, collagen matrix, a polylactic acid polymer or copolymer, a polyethylene glycol polymer or copolymer and chemical derivatives thereof may be included. When the pharmaceutical composition according to an embodiment is prepared in a formulation suitable for injection, the natural killer cell, the immune cell, or the substance increasing activity thereof may be dissolved in a pharmaceutically acceptable carrier, or may be frozen in the state of a solution where it is dissolved.

The pharmaceutical composition according to an embodiment may appropriately include, depending on the administration method or formulation, if needed, suspending agents, solubilizers, stabilizers, isotonizing agents, preservatives, adsorption inhibitors, surfactants, diluents, excipients, pH adjusters, analgesic agents, buffers, reducing agents, antioxidants, etc. Pharmaceutically acceptable carriers and formulations suitable for the present disclosure, including those described above, are described in detail in [Remington's Pharmaceutical Sciences, 19th ed., 1995] The pharmaceutical composition according to an embodiment may be formulated into a unit dosage form or placed in a multi-dose container by using a pharmaceutically acceptable carrier and/or excipient according to any method that is easily carried out by one of ordinary skill in the art. In this regard, the formulations may be in the form of solutions, suspensions, or emulsions in an oil or aqueous medium, or in the form of powders, granules, tablets or capsules.

Another aspect provides a method of preventing or treating a nervous system disease caused by nerve injury or abnormal nerves, the method including administering to a subject in need thereof an effective amount of an isolated natural killer cell, a cell population thereof, a substance increasing natural killer cell activity, or a combination thereof. In the method, the isolated natural killer cell, the immune cell, the cell population thereof, or the substance increasing activity of the natural killer cell or the immune cell, the nervous system disease caused by nerve injury or abnormal nerves, the preventing and treating, the administering are the same as described above.

Still another aspect provides use of an isolated natural killer cell, a cell population thereof, a substance increasing natural killer cell activity, or a combination thereof in preparing a pharmaceutical composition for preventing or treating a nervous system disease caused by nerve injury or abnormal nerves. In the use, the isolated natural killer cell, the immune cell, the cell population thereof, or the substance increasing activity of the natural killer cell or the immune cell, the nervous system disease caused by nerve injury or abnormal nerves, the preventing and treating are the same as described above.

Still another aspect provides a method of removing injured or abnormal nerves, the method including administering to a subject in need thereof an effective amount of an isolated natural killer cell, a cell population thereof, a substance increasing natural killer cell activity, or a combination thereof. In the method, the isolated natural killer cell, the immune cell, the cell population thereof, or the substance increasing activity of the natural killer cell or the immune cell, and the injured or abnormal nerves, the administering are the same as described above.

Still another aspect provides a method of killing injured or abnormal nerves, the method comprising contacting the injured or abnormal nerves with isolated natural killer (NK) cells or a cell population thereof.

The injured or abnormal nerves are the nerves expressing NKG2D ligands and the NK cells or the cell population thereof recognize and remove the injured or abnormal nerves via NKG2D receptor-ligand interaction through NK cell-mediated cytotoxicity.

The isolated NK cells or the cell population thereof are activated with cytokines, (e.g. IL-2), prior to the contact with the injured or abnormal nerves.

### ADVANTAGEOUS EFFECTS OF DISCLOSURE

According to a natural killer cell, an immune cell, or a substance increasing activity thereof according to an aspect, since the natural killer cell infiltrates into a nerve injury site to directly remove injured nerves, there is an effect of being usefully applied to fundamental treatment of a nervous system disease caused by nerve injury or abnormal nerves.

### BRIEF DESCRIPTION OF DRAWINGS

FIGS. 1A to 1E show results of examining cytotoxicity of IL-2-stimulated natural killer cells against embryonic DRG neurons in mice, wherein the mouse NK cells were isolated, enriched, and activated from adult mouse spleen, in which FIG. 1A shows results of intracellular staining of granzyme B of non-stimulated NK cells (left) and IL-2 stimulated NK cells (right), FIG. 1B shows results of flow cytometry for examining changes in NKp46⁺DX5⁺ NK cells in splenic lymphocytes by negative MACS enrichment (top-right quadrant), FIG. 1C shows result of immunostaining (β-tubulin III) of embryonic mouse DRG neurons after co-culture with IL-2 stimulated NK cells either by direct contact or separated by a transwell membrane, FIG. 1D shows results of ELISA detection of granzyme B in media of NK cell-DRG neuron co-cultures, and FIG. 1E shows results of examining effects of the NKG2D receptor on NK cell-mediated lysis of DRG neurons;

FIGS. 2A to 21 show results of examining susceptibility of cultured mouse embryonic DRG neurons to mouse NK-mediated cytotoxicity by RAE1, in which FIG. 2A shows immunolabeling of β-tubulin (magenta) and NKp46 (green) of embryonic DRG neurons or adult DRG neurons co-cultured with IL-2 stimulated NK cells, FIG. 2B shows results of LDH-release cytotoxicity assays of acutely cultured embryonic DRG neurons and adult DRG neurons at various Effector (NK): Target (DRG) (E: T) ratios, FIG. 2C shows images of in vitro time-lapse confocal Ca²⁺ imaging of rhodamine 3 AM-loaded embryonic DRG neurons or adult DRG neurons co-cultured with IL-2 stimulated NK cells isolated from adult male NKp46-YFP mice, FIG. 2D shows frequency over time of neurite Ca²⁺ events in embryonic DRG neurons or adult DRG neurons during IL-2 stimulated NK co-culture, FIG. 2E shows RT-PCR results of examining the expression of mRNA transcripts (Raet1, NK1.1, Advillin) in non-treated splenic NK cells, embryonic DRG neurons, or adult DRG neurons, FIG. 2F shows qRT-PCR results of examining Raet1 mRNA expression in embryonic DRG neurons or adult DRG neurons, FIG. 2G shows Western blot results of embryonic DRG neurons or adult DRG neurons with a pan-RAE1 antibody, FIG. 2H shows knockdown of Raet1 mRNA in embryonic DRG neurons using Raet1 selective siRNA, and FIG. 2I shows results of an LDH-release cytotoxicity assay of Raet1 mRNA knockdown embryonic DRG neurons;

FIGS. 3A to 31 show results of examining upregulation of Raet1 expression in injured adult mouse DRG neurons and mouse NK cell-mediated neurite fragmentation, in which FIG. 3A shows results of immunolabeling a neurite compartment with β-tubulin III after microfluidic culture of adult DRG neurons exposed to IL-2 stimulated NK cells, FIG. 3B shows results of quantification of DRG neurite density, FIG. 3C shows qRT-PCR results of examining time-dependent changes in Raet1 mRNA expression in adult DRG neuron cultures, FIG. 3D shows Western blot results of examining RAE1 protein expression in adult DRG cultures in vitro after 1 and 2 days, FIG. 3E shows results of β-tubulin III immunolabeling of an adult DRG culture alone (2 days in vitro) or an adult DRG culture co-cultured with IL-2 stimulated NK cells, FIG. 3F shows results of quantification of DRG neurite fragmentation, FIG. 3G shows results of knockdown of Raet1 mRNA in adult DRG neurons in vitro using Raet1 selective siRNA, FIG. 3H shows results of β-tubulin III immunolabeling of a Raet1 siRNA transfected adult DRG culture alone (2 days in vitro) or an adult DRG culture co-cultured with IL-2 stimulated NK cells, and FIG. 3I shows results of quantification of DRG neurite fragmentation;

FIGS. 4A and 4B show results of examining effects of blocking the NKG2D receptor of mouse NK cells on neurite degeneration of adult mouse DRG neurons, in which FIG. 4A shows results of β-tubulin III immunolabeling, after co-culturing anti-NKG2D antibody-pretreated adult DRG neurons with IL-2 stimulated NK cells, and FIG. 4B shows results of quantification of DRG neurite fragmentation;

FIGS. 5A to 5K show results of examining the effect of mouse peripheral nerve injury on Raet1 expression and the effect of injured sensory neurons on neurite fragmentation by stimulated mouse NK cells, in which FIG. 5A shows a schematic diagram of a spinal nerve transection site in relation to lumbar DRGs (L3, L4, and L5), FIG. 5B shows qRT-PCR results of examining time-dependent Raet1 mRNA expression in ipsilateral L5 DRG, after spinal nerve transection with respect to contralateral DRG, FIG. 5C shows qRT-PCR results of examining injury-related Raet1 mRNA expression in adult DRG neurons (1 day in vitro), FIG. 5D shows β-tubulin III immunolabeling of L5 DRG neurons isolated 7 days after co-culture (4 h) of sham DRG neurons with IL-2 stimulated NK cells, FIG. 5E shows results of quantification of DRG neurite fragmentation, FIG. 5F shows β-tubulin Ill immunolabeling of L5 DRG neurons isolated 7 days after DRG neurons subjected to spinal nerve transection were co-cultured (4 h) with IL-2 stimulated NK cells, FIG. 5G shows results of quantification of DRG neurite fragmentation, FIG. 5H shows results of anti-GFP immunolabeling of sciatic nerve tissue sections obtained from adult male NKp46-YFP mice after L5 spinal nerve transection injury, FIG. 5I shows results of quantification of the total number of YFP-positive events within a lymphocyte FSC/SSC gate from whole sciatic nerve homogenates, FIG. 5J shows results of ELISA quantification of granzyme B content in a whole sciatic nerve of a wild-type mouse after L5x injury, and FIG. 5K shows results of ELISA quantification of granzyme B content in a whole sciatic nerve of an NKp46-DTR mouse after L5x injury;

FIGS. 6A to 6C show results of characterization of NKp46-cre mice, in which FIG. 6A shows results of flow cytometry of peripheral blood lymphocytes from NKp46-YFP mice labelled with anti-NKp46 and anti-CD3 antibodies, FIG. 6B shows results of anti-GFP antibody (green) immunolabeling of spleen tissue sections from wild-type and NKp46-YFP mice, and FIG. 6C shows results of flow cytometry of peripheral blood lymphocytes from wild-type and NKp46-DTR mice 24 h after intravenous treatment with DTx (100 ng);

FIGS. 7A to 7E show results of examining effects of sciatic nerve crush on RAE1 in mouse peripheral nerve axons, in which FIG. 7A shows a schematic diagram of a sciatic nerve crush injury site in relation to lumbar DRGs (L3, L4, and L5), FIG. 7B shows qRT-PCR results of examining crush injury-related Raet1 mRNA expression in ipsilateral L3-5 DRG 3 days and 7 days post-surgery, FIG. 7C shows results of examining RAE1 protein expression in a sciatic nerve (3 days and 7 days) after peripheral nerve crush injury, FIG. 7D shows results of examining RAE1 protein expression in DRG neurons (3 days and 7 days) after peripheral nerve crush injury, and FIG. 7E shows results of examining RAE1 (green) co-localization with axonal marker β-tubulin III (magenta) in a sciatic nerve (3 days and 7 days) after tight ligation;

FIGS. 8A to 8G show results of examining effects of systemic NK cell depletion on sensory nerve injury after sciatic nerve crush in mice, in which FIG. 8A shows results of examining sciatic nerve tissue sections from adult male NKp46-YFP mice 7 days after sciatic nerve crush injury, FIG. 8B shows flow cytometry results of sciatic nerve homogenates obtained from adult male NKp46-YFP mouse 3 days after sciatic nerve crush injury, FIG. 8C shows results of quantification of the total number of CD45⁺/YFP⁺ double-positive events within a lymphocyte FSC/SSC gate from whole sciatic nerve homogenates, FIG. 8D shows results of examining changes in daily pin prick response score in NKp46-DTR mice administered intravenously with DTx every 4 to 5 days, FIG. 8E shows the area under the curve (AUC) in relation to cumulative sensation following full sciatic nerve crush and during recovery, FIG. 8F shows a heat map showing mean sensitivity to pin prick along the lateral hind paw, and FIG. 8G shows results of quantification of NKp46⁺/DX5⁺ double-positive lymphocytes in peripheral blood 16 days after sciatic nerve crush;

FIGS. 9A and 9B show the results of quantification of granzyme B in a whole sciatic nerve of mice after crush or sham injury, in which FIG. 9A shows the result of quantification of granzyme B in a whole sciatic nerve after crush or sham injury of wild-type mice, and FIG. 9B shows a result of quantification of granzyme B in a whole sciatic nerve after crush or sham injury of NKp46-DTR mice;

FIGS. 10A to 10H show the results of examining the effect of IL-2/anti-IL-2 antibody complex treatment on mouse NK cell-dependent acute sensory loss after partial sciatic nerve crush, in which FIG. 10A shows daily pin prick response scores for examining a transient reduction in sensitivity by treatment with an IL-2/anti-IL-2 antibody complex, FIG. 10B shows AUC values related to loss of cumulative sensation in mice treated with an IL-2/anti-IL-2 antibody complex, FIG. 10C shows a heat map showing mean sensitivity to pin prick along the lateral hind paw, FIG. 10D shows results of examining changes of NKp46⁺DX5⁺ NK cells, CD3⁺CD8⁺ T cells, or CD3⁺CD4⁺ T cells in peripheral blood by treatment with an IL-2/anti-IL-2 antibody complex, FIG. 10E shows daily pin prick response scores for examining a transient reduction in sensitivity by treatment with an IL-2/anti-IL-2 antibody complex in mice that have received anti-NK1.1 to deplete NK cells, FIG. 10F shows AUC values related to loss of cumulative sensation in mice treated with an IL-2/anti-IL-2 antibody complex, FIG. 10G shows a heat map showing mean sensitivity to pin prick along the lateral hind paw, and FIG. 10H shows results of examining changes of NKp46⁺DX5⁺ NK cells, CD3⁺CD8⁺ T cells, or CD3⁺CD4⁺ T cells in peripheral blood by treatment with an IL-2/anti-IL-2 antibody complex;

FIGS. 11A to 11C show results of examining the effect of NK cell depletion on the acute sensory loss by IL-2 complex treatment after partial sciatic nerve crush injury in mice, in which FIG. 11A shows daily pin prick response scores for examining a transient reduction in sensitivity by treatment with an IL-2/anti-IL-2 antibody complex in NKp46-DTR mice that have received a partial crush of the sciatic nerve with DTx treatment, FIG. 11B shows a heat map showing mean sensitivity to pin prick along the lateral hind paw, and FIG. 11C shows results of examining changes of NKp46⁺DX5⁺ NK cells, CD3⁺CD8⁺ T cells, or CD3⁺CD4⁺ T cells in peripheral blood by treatment with an IL-2/anti-IL-2 antibody complex;

FIGS. 12A to 12F show results of examining the effect of an IL-2/anti-IL2 antibody complex on long-term mechanical thresholds by axon injury in the sciatic nerve in mice, in which FIG. 12A shows results of β-tubulin III immunolabeling and quantification of full-length anisotropic sciatic nerve sections 6 days after partial crush in mice treated with an IL-2/anti-IL-2 antibody complex (a-proximal region, b-crush site, and c-distal region), FIG. 12B shows results of Stathmin 2 immunolabeling and quantification of full-length anisotropic sciatic nerve sections 6 days after partial crush in mice treated with an IL-2/anti-IL-2 antibody complex, FIG. 12C shows high-magnification images of β-tubulin III and STMN2 immunofluorescence in FIGS. 12A and 12B, respectively, FIG. 12D shows result of examining mechanical sensitivity thresholds of an ipsilateral hind paw 16 days after partial injury in mice treated with an IL-2/anti-IL-2 antibody complex, FIG. 12E shows result of examining mechanical sensitivity thresholds of an ipsilateral hind paw 15 days after partial injury and IL-2/anti-IL-2 antibody complex treatment of mice treated with anti-NK1.1 antibodies, and FIG. 12F shows results of examining a correlation between mechanical sensitivity outcomes in the injured limb and cumulative pin prick sensitivity;

FIG. 13 shows results of RAE1 (green) and STMN2 (magenta) co-immunolabeling in a chronically ligated sciatic nerve;

FIG. 14 shows results confirming that peripheral neuropathy caused by administration of an anticancer drug was induced since pain was induced on day 7 in an anticancer drug-administered group;

FIG. 15 shows results of examining the interaction between mouse NK cells and mouse peripheral sensory nerve cells which were cultured on day 7 after administration of the anticancer drug;

FIG. 16 shows results of examining ligand expression of NK cells in mouse sensory nerve cell tissues on day 7 after administration of the anticancer drug;

FIGS. 17A to 17C show images of selective infiltration of NK cells into brain tissues in which excitotoxicity of the central nervous system was induced in mice;

FIG. 18 shows results of analyzing behavioral responses occurring after induction of excitotoxicity and degeneration of the central nervous system in Nkp46-DTR transgenic mice;

FIG. 19 shows results of measuring ligand expression of NK cells in hippocampal tissues on day3 after induction of excitotoxicity and degeneration of the central nervous system in mice;

FIGS. 20A and 20B show results of analyzing behavioral responses following early administration of IL-2 activated mouse NK cells after partial crush injury in mice, in which FIG. 20A shows a schematic diagram of the early adoptive transfer after partial crush injury in mice, and FIG. 20B shows results of examining mechanical sensitivity thresholds of an ipsilateral hind paw in mice after the administration of IL-2 activated mouse NK cells;

FIGS. 21A and 21B show results of analyzing behavioral response following late administration of IL-2 activated mouse NK cells after partial crush injury in mice, in which FIG. 21A shows a schematic diagram of the late adoptive transfer after partial crush injury, and FIG. 21B shows results of examining mechanical sensitivity thresholds of an ipsilateral hind paw in mice during and after the administration of IL-2 activated mouse NK cells; and

FIGS. 22A to 22H show results of examining human NK cell-mediated neurite fragmentation in human iPSC (induced pluripotent stem cell) derived sensory neurons, in which FIG. 22A shows microfluidic human iPSC derived sensory neuron cultures, FIG. 22B shows flow cytometry results of IL-2 activated human NK cells labelled with anti-CD3, anti-CD56 antibodies, in which CD3⁻CD56⁺ cells were gated and analyzed for NKG2D expression, FIG. 22C shows flow cytometry results of IL-2 activated human NK cells labelled with anti-CD3 and anti-CD56 antibodies, in which CD3⁻CD56⁺ cells were gated and analyzed for perforin or granzyme B expression, FIG. 22D shows fluorescence images of iPSC derived sensory neurons stained against β-tubulin III in neurite compartment, and eight regions of interest (squares) are sampled for proximal (Prox.) and distal (Dist.) axon/neurite quantification, FIG. 22E shows distal axons/neurites from microfluidic devices treated with freshly thawed (control, left) or IL-2 primed human NK cells (right), and green indicates human NK cells, FIG. 22F shows quantification of axon/neurite fragmentation from 3 experimental repeats (6 microfluidic device; n=8 regions of interest (ROIs) per device) using NK cells from three different healthy donors and sensory neurons derived from iPSC donor ‘AD2’. Statistical analysis was performed using an unpaired t-test, with ns indicating p=0.1610 (t=1.717) and ** indicating p=0.0079 (t=4.931), FIG. 22G shows distal axons/neurites from microfluidic devices treated with freshly thawed (unstimulated) NK cells (control), IL-2 stimulated NK cells incubated with IgG1, or IL-2 stimulated NK cells incubated with anti-NKG2D. NK cells are shown in blue (DAPI) and axons are shown in magenta (β-tubulin III), and FIG. 22H shows the quantification of axon/neurite fragmentation from 3 experimental repeats (3 microfluidic device; n=10 ROIs per device). Microfluidic devices with different numbers of NK cells (50×10³, 100×10³, or 200×10³) were included for quantification. NK cells were obtained from three different healthy donors, and sensory neurons were derived from iPSC donor ‘840’. Multiple comparisons were analysed by two-way ANOVA with Šídák correction. Adjusted p=0.0106 for comparison between unstimulated NK cells and IL-2-stimulated NK cells with IgG1 at 100×10³ cells; adjusted p=0.0009 for comparison between unstimulated NK cells and IL-2-stimulated NK cells with IgG1 at 200×10³ cells; ** adjusted p=0.0036 for comparison between IL-2-stimulated NK cells incubated with IgG1 and anti-NKG2D at 200×10³ cells. Scale bars as indicated.

### MODE OF DISCLOSURE

Hereinafter, the present disclosure will be described in more detail with reference to exemplary embodiments. However, these exemplary embodiments are only for illustrating the present disclosure, and the scope of the present disclosure is not limited to these exemplary embodiments.

In the present disclosure, in vivo animal models were employed to demonstrate the therapeutic effects of natural killer cells on chronic neuropathic pain. However, due to limitations in directly evaluating cell-to-cell interactions between natural killer cells and human sensory neurons in animal models, in vitro microfluidic cultures of human induced pluripotent stem cell (iPSC)-derived peripheral sensory neurons were employed to examine whether the mechanism observed in animal models is conserved in human sensory neurons. These microfluidic culture systems offer a controlled neuronal microenvironment, enabling precise observation of neurite-level structural changes and facilitating a more detailed functional analysis of natural killer cell-mediated neurite fragmentation.

### Reference Example 1. Experimental Materials

All procedures were approved by the Institutional Animal Care and Use Committee (IACUC) at Seoul National University (Approval number: SNU-121011-1) and were reported in accordance with the ARRIVE guidelines (Kilkenny et al., 2010). Adult male and pregnant female wild-type C57BL/6 mice were purchased from Dae Han Bio Link (Taconic, South Korea). Rosa26eyfp (RRID:IMSR_JAX:006148) (Srinivas et al., 2001) and Rosa26dtr (RRID:IMSR_JAX:007900) (Buch et al., 2005) were purchased from Jackson Laboratories (USA). Ncr1icre mice (RRID:MGI:5308422) containing cre-recombinase inserted by homologous recombination at the 3′ end of Ncr1 (Nkp46) gene (Narni-Mancinelli et al., 2011) were a kind gift from Dr Eric Vivier. All mice were maintained as homozygous stocks in a specific pathogen free (SPF) facility. Double heterozygote Nrc1icre/wt;rosa26eyfp/wt (abbreviated to NKp46-YFP) and Nrc1icre/wt;rosa26dtr/wt (abbreviated to NKp46-DTR) mice were bred from single crosses in an SPF facility and transferred to a conventional room at least one week before experiments. Mice were maintained on a 12 h: 12 h light/dark cycle (lights on at 8:00 a.m.), housed 4-6 mice per cage on wood chip bedding and provided with standard laboratory feed and water ad libitum. DRG neurons and NK cells were prepared from male C57BL/6 mice (6-8 weeks); embryonic DRG neurons were prepared from embryos on day 15 in utero (E15) removed from euthanized female C57BL/6 mice. Nerve injury experiments were performed on male mice aged 7-9 weeks of indicated genotype. Animals were killed in accordance with Schedule 1 of the UK Animals (Scientific Procedures) Act 1986 by inhalation of a lethal concentration of isoflurane, followed by a lethal concentration of carbon dioxide for tissue culture.

### Reference Example 2. Experimental Procedures

### 2-1. Natural Killer Cell Depletion in Mice

NKp46-DTR mice were treated with diphtheria toxin (DTx; 100 ng) or sterile PBS solution (100 μL) intravenously by retro-orbital injection (Yardeni et al., 2011). Under isoflurane anaesthesia (3% induction, 1-2% maintenance in 100% O₂), an ophthalmic solution (0.5% proparacaine, Alcon, Belgium) was applied to one eye as a local anaesthetic. Insulin (0.3 mL; BD Biosciences) was inserted into the retro-orbital sinus of the eye and slowly injected. Injections were alternated between eyes. Injections of DTx or a sterile PBS solution administered blind to the content of the syringe starting one day prior to surgery and continuing every four to five days for the duration of the study. All mice were blood sampled at the end of experiments for depletion efficiency check by flow cytometry. For antibody depletion of NK cells, wild-type C57BL/6 mice were injected with 100 μg of LEAF purified anti-mouse NK1.1 (clone PK136) (Cat. no. 108712, RRID:AB_313399; Biolegend) or LEAF purified IgG2a, K isotype control (clone MOPC-173) (Cat. no. 400224, RRID:AB_326472; Biolegend) intravenously by retro-orbital injection one day before nerve injury.

### 2-2. Mouse L5 Spinal Nerve Transection (L5x)

Male mice aged 7-9 weeks were placed under isoflurane inhalation, the dorsal lumbar region was shaved, treated with an iodine solution (Potadine) and a unilateral incision made parallel to the L6 vertebrate. Under a ×20 dissection microscope illuminated by a cold light source, the musculature was parted by blunt forceps dissection to reveal the L6 transverse process, which was then cut and removed. The L5 spinal nerve, which runs immediately below the L6 process, was carefully freed of connective tissue and cut with fine spring scissors; 1 mm of the nerve was removed to prevent nerve regeneration. The wound was irrigated with sterile saline and closed in two layers with 6-0 silk sutures (Ailee, South Korea) and 9 mm skin clips (MikRon Precision, CA, USA). Mice were placed in a warm, darkened cage to recover from surgery.

### 2-3. Mouse Sciatic Nerve Crush

Adult male mice (7-9 weeks old) received a single unilateral crush injury to the sciatic nerve (Bridge et al., 1994). Briefly, under isoflurane anaesthesia, the right thigh was shaved and iodine treated and an incision was made mid-thing length. The sciatic nerve was exposed as it emerges from the sciatic foramen by parting the muscle with blunt forceps dissection. The nerve was carefully freed of connective tissue and fully crushed for 15 sec using fine, mirrorfinishe forceps (No 5; Dumont, Fine Science Tools, Germany). The wound was closed in two layers with two sutures of the overlying muscle facia and a single skin clip to close. Complete crush of the sciatic nerve was deemed successful by a sensory score of zero using the pin prick assay on the day following surgery; any mice with a pin prick response less than 24 h after surgery were excluded from analysis. For partial (moderate) crush, an ultra-fine haemostat (Cat. no. 13020-12; Fine Science Tools, Germany) was fitted with a custom spacer created from two layers of aluminium foil (15 μm thick) to create a gap 30 μm thick when the sciatic nerve was fully closed. The sciatic nerve was carefully free of connective tissue and placed between spacers on the haemostat (2-3 mm from the tip) by gently lifting the nerve using a fire-polished glassed rod. The haemostat was then closed on the first locking position and held for 15 sec before careful release of the nerve. The wound was closed in two layers with two sutures of the overlying muscle facia and a single skin clip. Mice were recovered in a warm, darkened cage. All tools were autoclaved prior to surgery and strict aseptic was maintained throughout.

### 2-4. IL-2/Anti-IL-2 Antibody Complex Treatment

Recombinant mouse IL-2 (Cat. no. 212-12, Lot no. 0608108; Peprotech, Rocky Hill, NJ, USA) was prepared as a stock at 0.1 mg/mL in PBS (without carrier protein) and stored at 4° C. for up to one week, according to the manufacturer's instructions. On the day of treatment, IL-2 (1.5 μg per mouse) was pre-mixed with anti-mouse IL-2 monoclonal antibody (50 μg per mouse) (S4B6-1 clone, RRID:AB_1107705; BioXCell) and incubated at room temperature for 15 min. The bound cytokine/antibody complex was further diluted in sterile PBS to a total volume in 500 μL and injected intraperitoneally. Injections were given once daily (evening time) for four days. For control experiments, mice were injected with an equal amount of rat IgG2a isotype peptide (clone 2A3, RRID:AB_1107769; BioXCell). The efficacy of the IL2/anti-IL-2 antibody complex treatment was confirmed by enlargement of the spleen compared to PBS injected mice one day after the final injection (Boyman et al., 2006).

### 2-5. Behavioral Testing

All sensory testing was performed between the hours of AM 9:00 and PM 18:00 in an isolated room maintained at 22±2° C. and 50±10% humidity. For mechanical threshold (von Frey filament) testing, mice were brought from the animal colony and placed in transparent plastic boxes on a metal mesh floor with 5×5 mm holes (Ugo Basile, Italy). The mice were then habituated for at least 30 min prior to testing. To assess mechanical sensitivity, the withdrawal threshold of the affected hind paw was measured using a series of von Frey filaments (0.20, 0.40, 0.70, 1.6, 3.9, 5.9, 9.8 and 13.7 mN; Stoelting, Wood Dale, IL, USA; equivalent in grams to 0.02, 0.04, 0.07, 0.16, 0.40, 0.60, 1.0 and 1.4). The 50% withdrawal threshold was determined using the up-down method as previously described (Chaplan et al., 1994). A brisk hind paw lift or flinch in response to von Frey filament stimulation was regarded as a withdrawal response. The 0.4 g filament was the first stimulus to be used, and when a withdrawal response was obtained, the next weaker filament was used. This process was repeated until no response was obtained, at which time the next stronger filament was administered. Interpolation of the 50% threshold was then carried out using the method of Dixon (Dixon, 1980). All behavioral testing was performed by an investigator who was blind to the treatment of the mice. Pin prick sensory recovery testing was performed as previously described (Ma et al., 2011) with slight modification. Mice were habituated on an elevated mesh in separate compartments. The lateral side of the affected hind paw was separated into five regions from the toe to the heel and stimulated with a stainless steel Austerlitz insect pin (Size 000; FST, Germany). A sensory response was confirmed by rapid lifting or flinching of the paw. The number of responses to two consecutive pin applications to the skin was recorded per region providing a score out of 10. Responses due to direct movement of the paw or hind limb (indicative of extraterritorial proprioception) were excluded. Testing was carried out daily until full sensory recovery (score of 10) was observed for all mice.

### 2-6. Mouse DRG Neuron Culture

Adult or embryonic DRG were rapidly dissected on ice-cold Ca²⁺- and Mg²⁺-free Hank's Buffer Saline Solution (HBSS, Welgene) (including 20 mM HEPES) and digested 30-60 min in collagenase A (1 mg/mL) and dispase II (2.4 U/mL) (Roche, Switzerland) at 37° C. Additional digestion was carried out for 5-7 min in trypsin (0.25%) and stopped with a trypsin inhibitor (2.5 mg/mL) (Cat. no. T9003; Sigma) in PBS followed by washing in Dubellco's Modified Eagle Medium (Gibco, Life Technologies) containing 10% serum (Gibco, Life Technologies). DRG were dissociated by trituration with a fire-polished glassed pipette in DMEM containing DNase I (125 U/mL) and centrifuged at 200 g on a layer of Bovine Serum Albumin (15% BSA solution; Sigma) before re-suspension in neurobasal medium (Gibco, Life Technologies) with B27 supplement, L-glutamine (1 mM), penicillin (100 U/mL) and streptomycin (100 U/mL) supplemented with nerve growth factor (NGF 2.5S) at 50 ng/ml. Adult DRG (10³ cells) and embryonic DRG (8×10³ cells) were plated on 10 mm diameter glass coverslips, glass bottom dishes or 96 well flat-bottom culture plates (Nunclon, Thermo Scientific) previously coated with poly-D-lysine (10 μg/mL) and laminin (10 μg/mL) (Sigma). For injured DRG experiments, ipsilateral L5 DRG were rapidly dissected on ice-cold HBSS from adult mice 7 days after L5x or sham surgery, pooled (n=3 DRG per group) and dissociated to a single cell suspension as above. DRG were then seeded onto poly-D-lysine and laminin-coated glass bottom dishes (10³ cells per dish) and cultured overnight in neurobasal medium containing NGF (50 ng/ml). For microfluidic cocultures, DRG neurons isolated from adult mice (as above) were suspended in neurobasal medium and seeded (10⁴ cells) into the somal reservoir of a microfluidic device (Xona Microfluidics, CA, USA) previously coated with poly-D-lysine (10 μg/mL) and laminin (10 μg/mL) (Sigma). NGF (100 ng/ml) was added to the media in the neurite reservoir. Mouse neurons were cultured for 5 days during which neurites grew along 3 μm×500 μm channels connected to the neurite reservoir.

2-7. Human iPSC (Induced Pluripotent Stem Cell) Derived Sensory Neuron Culture

Human iPSCs (hiPSCs) from healthy control donors were obtained via the University of Oxford StemBANCC consortium. hiPSC were maintained in 6-well culture plates coated with hESC-qualified LDEV-free Matrigel (Cat. no. 354277; Corning) with daily changes of mTeSR1 media (Cat. no. 85850; StemCell Technologies). Cells were routinely passaged by EDTA dissociation (500 μM in PBS, 5 min) when cells became 70-80% confluent. hiPSCs were differentiated into sensory neurons in 6 well plates using a combination of small-molecule dual SMAD (mothers against decapentaplegic family transcription factor) inhibitors SB431542 (10 μM) (Cat. no. 1614; Tocris) and LDN-193189 (100 nM) (Cat. no. 6053; Tocris) and Wnt pathway activators CHIR99021 (3 μM) (Cat. no. 4423; Tocris), SU5402 (10 μM) (Cat. no. SML0443; Sigma), and DAPT (10 μM) (Cat. no. 2634; Tocris). Briefly, hiPSC media was changed to Mouse Fibroblast-conditioned media (MEF) (Cat. no. AR005; R&D Systems) supplemented with Fibroblast growth factor (FGF)-2 (10 ng/ml) for one day (Day-1). The next day (Day 0) differentiation began with the addition of a series of small molecule inhibitors added to Knock-out Serum Replacement (KSR) media: Knockout DMEM (Cat. no. 10829018; Life Technologies) containing 15% knockout-serum replacement (Cat no. 10828010; Life Technologies), 1% Glutamax, 1% non-essential amino acids, 1% antibiotic/antimycotic, and β-mercaptoethanol (100 μM). Media was changed daily (2 mL per well), during which time KSR media was gradually replaced with N₂ ‘complete’ media (neurobasal medium supplemented with N₂ (Cat. no. 17502-048), B-27 minus vitamin A (Cat. no. 12587-010; Gibco), Glutamax (Cat. no. 35050-038; Gibco) and 1× antibiotic-antimycotic (Anti-anti) (Cat. no. 15240-062; Gibco) plus recombinant human β-NGF (rhNGF) (Cat. no. 450-01; Peprotech), NT3 (Cat. no. 450-03; Peprotech), GDNF (Cat. no. 450-10; Peprotech), and BDNF (Cat. no. PHC7074; Life Technologies) at 25 ng/mL each), according to published protocols. On day 11 or 12 of differentiation, cells were washed with 1 mL per well of PBS, followed by knockout DMEM containing DNase I (100 U/mL) and incubated 5 min at 37° C., 5% CO₂. The knockout DMEM was then replaced with 1 mL TrypLE dissociation reagent and incubated for a further 5 min at 37° C., 5% CO₂. before dissociation with fire-polished glass pipette. Cells were transferred to PBS in 15 mL tubes and centrifuged 5 min, 500×g to pellet. Supernatant was removed and cells were resuspended in N2 ‘complete’ media supplemented with CHIR99021 (3 μM) and Rho kinase inhibitor, Y-27632 (10 μM) (Cat. no. 1254; Tocris). Cells were counted, suspension at 4×10⁶ cells per mL in N2 completed neuronal media (above), diluted 1:1 with chilled 2× freeze media (20% DMSO, 80% charcoal-stripped FBS) and immediately transfer cell suspension to cryovials (2×10⁶ cells per mL per vial).

Thawed precursor neurons were seeded either onto 13 mm diameter glass coverslips (approximately 20,000 cells per coverslip) or microfluidic devices (50,000 cells per device) previously coated with poly-D-lysine (PDL) (10 μg/mL) followed by reduced growth-factor Matrigel (Cat. no. 354277; Corning) or Geltrex (LDEV-Free, hESC-Qualified) (Cat. no. A1413302; ThermoFisher). Sensory neuron precursors were maintained in N2 ‘complete’ media supplemented with CHIR99021 (3 μM) and Rho kinase inhibitor, Y-27632 (10 μM) for the first two days after seeding, after which the neuronal media was supplemented with cytosine arabinoside (Ara-C) (0.5-1 μM) (Cat. no. C1768; Sigma) to remove proliferating, non-neuronal cells. Neurons were matured in N2 ‘complete’ media in an incubator at 37° C., 5% CO₂ for at least 4 weeks (40 days after differentiation), with media changes twice per week.

### 2-8. Natural Killer Cell Isolation and Stimulation

### 2-8-1. Mouse NK Cell Isolation and Stimulation

Mouse NK cells were prepared from spleens of adult male C57BL/6 mouse (6-8 weeks old). Spleen cells were homogenized by sequentially passing through 70 μm and 40 μm cell strainers (Falcon, BD Biosciences). Red blood cells were lysed by incubation for 2 min in ACK lysis buffer (in mM: 150 NH₄Cl, 10 KHCO₃, 0.1 Na₂EDTA, pH 7.3). Single-cell suspensions were then passed through nylon wool columns (Polysciences, Warrington, PA) for the depletion of adherent populations, consisting of B cells and macrophages. Eluted cells were resuspended in 0.01 M phosphate buffered saline (PBS)+2 mM EDTA and 2% FBS. Mouse NK cells were enriched using a magnetic associated cell sorting (MACS) method in combination with a negative selection protocol (Mouse NK cell isolation Kit II, Cat. no. 130-096-892; Miltenyi Biotec GmbH) according to the manufacturer's instructions. Briefly, cell suspensions were sequentially incubated at 4° C. with a cocktail of biotin-conjugated monoclonal antibodies against non-NK cells followed by anti-biotin microbeads. The cell suspension was then passed through an LS column placed in a magnetic field (MidiMACS Separator; Miltenyi Biotec GmbH). Bead-conjugated non-NK cells remained in the columns while non-labelled NK cells passed through in the eluent. Enriched NK cells were either used directly (control) or stimulated with recombinant murine interleukin (IL)-2 (Cat. no. 212-12, Lot no. 0608108; Peprotech) at 1000 U/mL for two days before use in experiments. Mouse NK cells were cultured at 2×10⁶ cells per mL in RPMI 1640 medium (Gibco, Life Technologies) supplemented with fetal bovine serum (FBS) (10%) and penicillin/streptomycin (100 U/mL) in 96-well U-bottomed (Falcon, BD Biosciences) plates for 48 h. The purity of mouse NK cells (NKp46⁺DX5⁺) in the eluent was checked by flow cytometry to be consistently above 90%. The cells were then harvested and used as effector cells in co-culture and cytotoxicity experiments.

### 2-8-2. Human NK Cell Isolation and Stimulation

Human NK cells were prepared from the peripheral blood of three healthy volunteers, as well as leukocyte cones from three volunteer blood donors supplied by UK National Health Service Blood and Transplant (NHSBT) Non-Clinical Issue (NCI) service.

Peripheral blood was collected by venepuncture in sodium-heparin tubes. Whole blood was then diluted 1:1 in serum-free RPMI 1640 media (Cat. no. 21875034; Life Technologies) and suspended on 15 mL Lympholyte Human Cell Separation Media (Cat. CL5020, CedarLane) in 50 mL SepMate tubes (Cat. no. 85450; Stem Cell Technologies) and centrifuged 22 min at 800×g at room temperature (RT) with no brake to separate peripheral blood mononuclear cells (PBMCs). Plasma containing platelets was removed and the leukocyte layer transferred to fresh RPMI containing 10% fetal bovine serum (heat-inactivated) (Cat. no. F9665-500ML; Sigma-Merck) and washed by pellet centrifugation at 500×g, 10 min and RT. Live PBMC were counted by trypan blue exclusion and 1×10⁸ cells per sample were transferred to magnetic cell sorting (MACS) buffer (0.01M phosphate buffered saline (PBS) plus 2 mM EDTA and 2% FBS). NK cells were enriched by MACS negative selection using a human NK Cell Isolation Kit (Cat. no. 130-092-657; Miltenyi Biotec) with LD columns (Cat. no. 130-042-901; Miltenyi Biotec), according to the manufacturer's instructions. Unlabelled NK cells were eluted into MACS buffer and counted with trypan blue exclusion for downstream applications. 4.65-6.75×10⁶ live NK cells were isolated per 1×10⁸ PBMC per donor. Approximately 1×10⁶ purified NK cells were sampled from each donor for purity check by flow cytometry analysis. Remaining NK cells were suspended at 1×10⁶ cells/mL in cryopreservation media (50% RPMI, 40% FBS, 10% DMSO) and frozen using a controlled-rate alcohol-free cell freezing container (CoolCell, Corning) before transfer to vapour phase nitrogen for long-term cryostorage.

Cone blood was diluted 1:1 in PBS containing 1% BSA and suspended on 15 mL of Lympholyte® Human Cell Separation Media (Cat. no. CL5020; CedarLane) in 50 mL Falcon tube and centrifuged 22 min at 800×g (slow acceleration [3] and deceleration [0]) at RT to separate PBMCs. PBMCs located in the interphase (‘buffy coat’) between Lympholyte® and plasma layers were carefully collected into a new 50 mL Falcon tube and then washed by MACS buffer by pellet centrifugation 10 min at 400×g at room temperature. 100×10⁶ PBMCs were resuspended in 2 mL of MACS buffer. NK cells were enriched by negative selection using EasySep™ Human NK Cell Isolation Kit (Cat. no. 17955; Stem Cell), according to the manufacturer's instructions. The isolated NK cells were suspended in cryopreservation media (50% RPMI, 40% FBS, 10% DMSO) and frozen using a controlled-rate alcohol-free cell freezing container (CoolCell, Corning) in −80° C. before being transferred to vapour phase nitrogen for long-term cryostorage. For NK cell stimulation, vials of cryopreserved NK cells were rapidly thawed in a water bath at 37° C. and washed in RPMI including 10% FBS supplemented with DNase I (125 U/mL) followed by centrifugation at 400×g, 10 min, RT. Cells were counted and seeded at 2×10⁶ cells per mL in RPMI+10% FBS in 96 well U-bottom plates (Nunclon Delta, Cat. no. 10344311; FisherScientific) supplemented with recombinant human IL-2 (103 U/mL; 100 ng/ml equivalent) (Cat. no. 200-02; Peprotech) and cultured for 2 days at 37° C., 5% CO₂. The purity of NK cells (CD3⁻CD56+) was examined by flow cytometry to be consistently above 90%. Key markers associated with NK cell activation and cytotoxic function (NKG2D, Granzyme B, and Perforin) were also expressed in more than 90% of the CD3⁻CD56+ population.

### 2-9. Mouse DRG-Mouse NK Co-Cultures

Control or IL-2 stimulated mouse NK cells were harvested, washed in RPMI and resuspended in neurobasal media. Mouse DRG were washed once in neurobasal media (NGF-free) and mouse NK cells were added to the neurite compartment (5×10⁵ cells) for microfluidic cultures or seeded (2.5×10⁵ cells) directly over the DRG for glass coverslip cultures, and co-cultured for 4 h at 37° C. and 5% CO₂. Co-cultures were carefully washed once in warm HBSS and fixed with 2-4% PFA in 0.01 M PBS (pH 7.4) for 30 min at room temperature, followed by washing in PBS (3×10 mins) at storage at 4° C. prior to immunolabeling for β-tubulin III and NKp46 (see Immunofluorescence). For trans-well experiments, glass coverslip cultures of DRG were transferred to a 24-well plate in 500 μL neurobasal media. Mouse NK cells (2.5×10⁵ per well) were seeded onto 6.5 mm diameter polycarbonate trans-well membrane inserts with 0.4 μm pore size (Corning) and incubated for 4 h at 37° C. and 5% CO₂ before removal of the membrane and fixation as above. For antibody blocking of NKG2D function, mouse NK cells were incubated with 30 μg/mL of LEAF purified anti-mouse CD314 (NKG2D) (CX5 clone) (Cat. no. 130204, RRID:AB_1227715; Biolegend) or LEAF purified rat IgG1 isotype control (Clone RTK2071) (Cat. no. 400414. RRID:AB_326520; Biolegend) for 15 min at room temperature (2.5×10⁶ NK cells per mL) before addition to target mouse DRG neurons.

2-10. Human iPSC-Derived Sensory Neuron-NK Cell Co-Cultures

Control or IL-2 stimulated human NK cells were harvested, washed in PBS and resuspended in ‘complete’ neurobasal (NB) media. Human NK cells were added to the neurite compartment (5×10⁵ cells) for microfluidic cultures, and co-cultured for 4 h at 37° C. and 5% CO₂. Co-cultures were carefully washed once in warm HBSS and fixed with 2-4% PFA in 0.01 M PBS (pH 7.4) for 30 min at room temperature, followed by washing in PBS (3×10 mins) at storage at 4° C. prior to immunolabeling for β-tubulin III (axon/neurite) and NKp46 (NK cell) (see immunofluorescence).

### 2-11. LDH-Release Cytotoxicity

Effector NK cells were assayed for cytotoxicity against DRG neuron targets by measuring the release of lactate dehydrogenase (LDH) into the culture medium using an LDH Cytotoxicity Assay Kit (Thermo Scientific Pierce). Control or IL-2 stimulated NK cells were harvested, washed in RPMI and added to DRG cultures in 96 well plates (Nunclon, Thermo Scientific) in neurobasal medium at various ratios and cultured for 4 h before sampling of media supernatant which was assayed for LDH activity according to manufacturer's instructions. Absorbance values were acquired on a microplate spectrophotometer (BioTek Instruments). Each ratio was determined in triplicate. Specific cytotoxicity was calculated as follows: % cytotoxicity=[(experimental release-spontaneous release)/(maximum release-spontaneous release)]×100. The optimum number of target cells (DRG neurons) for maximum LDH release detection was determined prior to experiments (8×10 embryonic DRG neurons; 103 adult DRG neurons).

### 2-12. Live Confocal Imaging

Adult or embryonic mouse DRG (1×10³ and 8×10³ neurons, respectively) cultured for one day on PDL and laminin-coated glass-bottom petri dishes were fluorescently labelled with Vybrant Dil (Cat. no. V-22886; Molecular Probes) or loaded with the Ca²⁺ indicator rhodamine 3-AM (Cat. no. R10145; Molecular Probes) according to the manufacturer's instructions. NK cells previously isolated from NKp46-YFP mice and stimulated IL-2 (1,000 U/mL) for 48 h were suspended in neurobasal media and seeded onto the coverslip (2.5×10⁵ cells per dish). Dishes containing mouse DRG-mouse NK co-cultures were immediately transferred to a confocal microscope (LSM 700; Zeiss) and maintained in a humidified atmosphere at 37° C. and 5% CO₂ (Live Cell Instruments, Seoul, South Korea). A time series of single z-section images (512×512) were acquired using a multitrack setting (488 nm and 555 nm fluorescence emission and differential interference contrast (DIC) bright-field) at 30-60 sec intervals and multiple positions under the control of Definite Focus. Images were acquired up to 3 h and exported as a sequential time-lapse in AVI format.

### 2-13. In Vivo Two Photon Sciatic Nerve Imaging

Male NKp46-YFP mice (8-9 weeks old) received unilateral sciatic nerve crush or sham surgery. On day 3, mice were anaesthetized with pentobarbital (80 mg/kg, i.p.) supplemented with 20 mg/kg immediately prior to recording. Some mice additionally received Dextran-Texas red (neutral 40,000 m.w.) (Molecular Probes) given via retro-orbital injection (100 μL, 10 mg/mL) to visualize the vasculature and confirm maintenance of blood flow during recording. The sciatic nerve was re-exposed and bathed in sterile saline. Mice were placed on a warm pad maintained at 35° C. for the duration of the imaging. The sciatic nerve was carefully lifted with two curved glass rods held via a micromanipulator at both distal sides of the exposed nerve to mechanically isolate the nerve from the body and minimize the breathing-induced motion artefacts. A W plan-Apochromat 20× water immersion lens was lowered to the nerve surface 1-2 mm distal from the crush site to identify the blood vessels and YFP-positive cells. After the Ti-sapphire laser (Chameleon, COHERENT) was tuned to the wavelength of 900 nm for excitation of both YFP and Texas Red, 3D time-lapse imaging was performed using a laser scanning confocal microscope (LSM 7MP; Zeiss) as follow: 1024×512 pixels per a single image, 0.79 μs of pixel dwell, 2 μm×25 sections per a z-stack (total 50 μm depth) at 30 sec interval. The two-photon laser power was compensated according to the depth across 5-8% of total power. The image was rendered into 2D video using imaging software (Zeiss Efficient Navigation 2012).

### 2-14. Mouse DRG Small Interference RNA Gene Knockdown

Acute isolated adult or E15 embryonic mouse DRG were transfected with siRNA by electroporation (Neon, Invitrogen) according to the manufacturer's instructions. Briefly, single cell suspensions of mouse DRG were suspended in an electroporation medium containing siRNA oligonucleotide and drawn into a tip containing a gold-plated electrode (5×10⁴ cells per 10 μL). Tips of cells were placed into a tube containing electrolytic buffer (1,500 V, 20 ms), and electroporated (1,500 V, 20 ms), and immediately ejected into penicillin/streptomycin-free neurobasal media containing 50 ng/ml NGF and cultured for 48 h at 37° C., 5% CO₂ until assessment of assessment of knockdown or functional experiments. Prior to experiments, transfection efficiency was optimized by electroporation of DRG with a cDNA plasmid encoding green fluorescent protein and examination of GFP fluorescence after two days in culture. Two siRNA oligonucleotides per target were tested for knockdown efficiency by real time PCR; siRNA oligos that reduced mRNA expression by more than 70% compared to a negative control siRNA oligonucleotide were used for functional experiments. Gapdh siRNA oligonucleotides (10 nM) (Silencer Select, Cat. no. 4390849; Ambion, Life Technologies) were used as positive control.

2-15. Peripheral Blood Lymphocyte Isolation from Mice

Systemic depletion of NK cells was confirmed at the end of experiments in a sample of peripheral blood obtained by retro-orbital bleed. Briefly, under isoflurane anaesthesia, a 15 mL glass Pasteur pipette was inserted into the retro-orbital sinus and gently twisted to disrupt the orbital venous plexus. 50 to 100 μL of blood was removed by capillary action and ejected into heparin-coated tubes (Idexx Laboratories, USA). Blood samples were diluted with an equal volume of serum-free RPMI, floated on top of a lymphocyte separation medium (Lympholyte® Mammal, Cedarlane Labs) and centrifuged at 1000×g for 20 min with a slow acceleration gradient. Peripheral blood mononuclear cells were collected from the monolayer, washed in RPMI and suspended in FACS buffer (5% FBS, 0.002% NaN₃ in 0.01 M PBS) for flow cytometry analysis.

### 2-16. Flow Cytometry

For surface labeling, cell suspensions were transferred to 5 mL round bottom tubes and Fc receptors were blocked with unconjugated rat anti-mouse CD16/CD32 (clone 2.4G2) monoclonal antibody (1:100; Cat. no. 553142, RRID:AB_394657; BD Biosciences) for 15 min at 4° C. and labelled with a combination of fluorescently conjugated anti-mouse primary antibodies for 30 min at 4° C. After washing with FACS buffer, cell suspensions were run on a four-colour flow cytometer (FACSCalibur, BD Biosciences) and gated populations analyzed with Cell Quest software (BD Biosciences). Lymphocytes were initially gated according to the FSC-SSC scatter profile; cell populations were identified by fluorescence gating compared to unlabeled or IgG controls. Antibodies used were PE rat anti-mouse NKp46 (clone 29A1.4) monoclonal antibody (1:200; Cat. no. 12-3351, RRID:AB_1210743; eBioscience), APC rat anti-mouse CD49b (clone DX5) monoclonal antibody (1:500; Cat. no. 17-5971, RRID:AB_469484; eBioscience), FITC Armenian hamster anti-mouse CD3e (clone 145-2C11) monoclonal antibody (1:1,000; Cat. no. 11-0031, RRID:AB_464881; eBioscience), APC rat anti-mouse CD45 (clone 30-F11) monoclonal antibody (1:200; Cat. no. 17-0451, RRID:AB_469393; eBioscience), PE rat anti-mouse CD4 (clone GK1.5) monoclonal antibody (1:1,000; Cat. no. 12-0041, RRID:AB_465507; eBioscience), APC rat anti-mouse CD8a (clone 53-6.7) monoclonal antibody (1:1,000; Cat. no. 17-0081, RRID:AB_469335 eBioscience), FITC CD3 (1:50; Cat. no. 300452, RRID:AB_2564148; BioLegend), BV786 CD56 (1:50; Cat. no. 564058; RRID:AB_2738569; BD Biosciences, PE NKG2D (1:50; Cat. no. 130-111-646; Miltenyi Biotec), PE granzyme B (1:50; Cat. no. 561142, RRID:AB_10561690; BD Biosciences), BV421 perforin (1:50; Cat. no. 353307; RRID:AB_11149688; BioLegend). Titres of flow cytometry antibodies were determined prior to experiments by comparison to equivalent concentrations of fluorescence-conjugated IgG isotype controls. Percentages of peripheral blood cell populations were calculated from 20,000 gated lymphocyte events.

### 2-17. Intracellular Staining

For intracellular labeling of granzyme B, cell suspensions were washed in RPMI, resuspended in FACS buffer, and Fc receptors were blocked for 15 min at 4° C. Cells then underwent fixation and permeabilization in BD Cytofix/Cytoperm buffer (BD Biosciences, cat no. 554714) for 20 min at 4° C. followed by labeling with PE rat anti-mouse granzyme B (clone NGZB) monoclonal antibody (1:100; Cat. no. 12-8898, RRID:AB_10853811; eBioscience) or PE rat IgG2a isotype control (1:100; Cat. no. 12-4321, RRID:AB_470052; eBioscience) for 30 min at 4° C. and washing in BD Perm/Wash buffer.

### 2-18. Preparation of Mouse Sciatic Nerve Suspensions and Flow Cytometry

For flow cytometry analysis of NK cells present in sciatic nerve, NKp46-YFP mice were deeply anaesthetized with pentobarbital (100 mg/kg, i.p.) followed by trans-cardiac perfusion with PBS (0.01 M, pH 7.4) at various time points after peripheral nerve injury to remove peripheral blood from the circulation. Bilateral sciatic nerves were rapidly removed to ice-cold Ca²⁺- and Mg²⁺-free HBSS (Welgene) (including 20 mM HEPES) and cut into 1-2 mm pieces. Tissues were transferred to 15 mL tubes and centrifuged at 500×g for 5 min. HBSS was replaced with collagenase A (1 mg/mL) and dispase II (2.4 U/mL) (Roche, Switzerland) and incubated for 90 min at 37° C. with frequent gentle agitation. Additional digestion was carried out for 5 min in trypsin (0.25%) and stopped with a trypsin inhibitor (2.5 mg/mL) (T9003; Sigma) in PBS followed by washing in RPMI containing 10% serum (Gibco, Life Technologies). Nerves were dissociated by trituration with a fire-polished glassed pipette in RPMI containing DNase I (125 U/mL), passed through a 30 μm separation filter to remove debris (Miltenyi Biotec) and resuspended in FACS buffer in 5 mL round bottom tubes. Prior to sciatic nerve flow cytometry, CD45⁺ lymphocyte and NKp46-YFP cell gating was set on peripheral blood lymphocytes from wild-type and NKp46-YFP mice. Sciatic nerve samples were run on the slow setting until the total event count was less than 50 per second, or gated events were less than one per 5 sec. NK cells were identified by lymphocyte FSC-SSC scatter profile and YFP fluorescence; in some experiments, total lymphocytes were additionally labelled with an APC-conjugated anti-mouse CD45 antibody (1:400; Cat. no. 17-0451, RRID:AB_469393; eBioscience).

2-19. Tissue Preparation from Mice

At specific time points after mouse nerve injury or sham surgery, mice were deeply anaesthetized with pentobarbital (100 mg/kg, i.p.) followed by exsanguination by transcardiac perfusion with PBS (0.01 M, pH 7.4) containing heparin (500 U/L). Whole DRG (lumbar L3-L5) and full-length sciatic nerve (spinal nerves to peripheral trifurcation) tissues were dissected and collected in sample tubes which were immediately frozen on liquid nitrogen and stored at −70° C. for later molecular analysis. For immunohistochemistry, PBS perfusion was followed by a paraformaldehyde fixative (4% PFA, 0.2% picric acid in 0.1 M PBS, pH 7.4). Sciatic nerves were post-fixed up to 24 h and cryoprotected in sucrose solution (30% sucrose in 0.01 M PBS, pH 7.4) at 4° C. Fixed sciatic nerve tissues were embedded in frozen section compound (Optimal Cutting Temperature; Leica), sectioned on a cryostat (14 μm) and thaw-mounted on glass microscope slides (Superfrost Plus; Fischer Scientific).

### 2-20. Immunofluorescence

After washing with PBS (3×10 min), cells and tissues were blocked and permeabilized in 0.1-0.3% triton-X 100 and 10% normal donkey serum (NDS, Jackson ImmunoResearch) in PBS for 1 h at room temperature. Primary antibodies were applied in 1% NDS, 0.01-0.03% triton-X 100 in PBS and incubated overnight at 4° C. in a humidified chamber. Primary antibodies used were as follows: Rabbit anti-β-tubulin III (1:400-500; Cat. no. T2200, RRID:AB_262133; Sigma), goat anti-NKp46 (1:200; Cat. no. AF2225, RRID:AB_355192; R&D Systems), rabbit anti-STMN2 (1:500; Cat. no. NBP1-49461, RRID:AB_10011569; Novus Biologicals), goat anti-mouse pan-RAE1 antibody (1:40; Cat. no. AF1136, RRID:AB_2238016; R&D systems), and Goat anti-NKp46/NCR1 antibody (1:200; Cat. no. AF1850; R&D systems). Fluorescence conjugated secondary antibodies were incubated in 1% NDS, 0.1-0.3% triton-X 100 in PBS for 1 h at RT the dark. Secondary antibodies used were as follows: Alexa Fluor 647 donkey anti-rabbit (1:200; Cat. no. A31573, RRID:AB_2536183; Molecular Probes) and Alexa Fluor 488 donkey anti-goat (1:200; Cat. no. 705-545-003, RRID:AB_2340428; Jackson ImmunoResearch). For RAE1 double labeling with β-tubulin III and STMN2, primary and secondary labeling was performed sequentially for each antibody with a repeat blocking stage. Fluorescence images (1024×1024) were acquired sequentially on a laser-scanning confocal microscope (LSM 700 Zeiss) with Zen 2012 software (v8.1 SP1, Zeiss). Coverslips were imaged in a single z-section. Tissue sections were mounted with DAPI-containing hardsetting aqueous mounting medium (Vectorsheild, Vector Laboratories) and imaged as a stack of 4-5×2 μm z-sections and exported as a maximum intensity projection TIFF.

### 2-21. Western Blot

Tissues and cells were homogenized in RIPA buffer (Cat. no. 20-188; Millipore) containing protease inhibitor cocktail (Cat. no. P8340; Sigma) and phosphatase inhibitor cocktail (Cat. no. P3200; Gendepot). Cultured cells were washed with warmed HBSS and collected in protein lysis buffer by scraping; frozen tissues were disrupted either in a Minilys bead homogenizer (Precellys) or glass grinder. Homogenized samples were then sonicated (3×10 s, 25% amplitude) on ice and then spun at high speed (10,000×g) for 10 min at 4° C. after 40 min incubation on ice and pellet discarded. An equal volume of 5x SDS sample buffer was added to the sample lysates which were boiled at 9° C. heat block for 5 min. Protein content was determined by colorimetric assay (Lowry, BioRad). Equal amounts of protein (25 to 40 μg) and protein size markers were separated by SDS-polyacrylamide gel electrophoresis (5% stacking gel, 10% resolving gel) followed by transfer to a PVDF membrane. Membranes were blocked in a 5% skimmed milk solution containing Tris buffered saline and 0.1% tween-20 (TBS-T) at room temperature for 1 h and subsequently incubated with goat anti-mouse Pan-RAE1 antibody (1:500; Cat. no. AF1136, RRID:AB_2238016; R&D systems) overnight at 4° C. in blocking solution. Blots were washed with TBS-T (3×10 min) and then incubated with anti-goat HRP-conjugated secondary antibody (1:10,000; Cat. no. 2020, RRID:AB_631728; Santa Cruz) for 1 h at room temperature. After washing with TBS-T, blots were developed by application of western ECL substrate (Cat. no. 1705061; BioRad) according to the manufacturer's instructions and images of sequential exposure times were acquired digitally (ChemiDoc; BioRad). Blots were then stripped with stripping buffer at 50° C. for 30 min followed by TBS-T washing and subsequently incubated with mouse anti-beta-actin (1:10,000; Cat. no. A5441, RRID:AB_476744; Sigma) and rabbit anti-N-cadherin (1:10,000; Cat. no. 04-1126, RRID:AB_1977064; Millipore) in blocking solution. Goat anti-mouse polyclonal (1:10,000; Cat. no. K-0211589, RRID:AB_2636911; Komabiotech) or goat anti-rabbit (1:10,000; Cat. no. sc-2004, RRID:AB_631746; Santa Cruz) HRP-conjugated secondary antibodies were applied, respectively, and then exposed after ECL treatment. Embryonic mouse head tissue was used as positive control for RAE1.

### 2-22. Enzyme-Linked Immunosorbant Assay (ELISA)

Granzyme B content of mouse sciatic nerves was determined by ELISA according to the manufacturer's instructions (DuoSet, Cat. no. DY1865; R&D Systems). Briefly, 96 well plates were coated with capture antibody overnight at room temperature, washed, and blocked 1 h. Frozen tissues were homogenized on a shaker with 1.4 mm zirconium beads (Precellys) in RIPA lysis buffer (Millipore) and lysates were centrifuged 13,000 rpm for 5 min. Supernatants were diluted 1:1 with reagent diluent, added in duplicate to coated wells along with a granzyme B standard series and incubated at room temperature for 2 h. Plates were washed thoroughly before addition of Streptavidin-HRP, followed by substrate buffer. Stop solution was applied and absorbance (450 nm minus 540 nm correction) read on a microplate spectrophotometer (BioTek Instruments, VT, US). Granzyme B levels in samples were analyzed using the four-parameter logistic curve fitting algorithm by reference to standard curve values and performed using online software (http://www.elisaanalysis.com/).

### 2-23. Reverse Transcription Polymerase Chain Reaction (RT-PCR)

Total RNA was extracted from L5 DRG from L5x or sham mice (two mice pooled per sample) and L3-L5 DRG from sciatic crush or sham mice (one mouse per sample). Tissues were disrupted in RTL Plus lysis buffer (Qiagen) including 1% β-mercaptoethanol with a mini glass mortar and pestle on ice and further homogenized using a Minilys bead homogenizer (Precellys, Bertin, France); DRG cultures were washed once in warm HBSS prior to lysis by pipetting; frozen cell pellets were vortexed in lysis buffer. RNA was purified from lysed samples, including genomic DNA elimination, by on-column extraction (RNeasy Plus; Qiagen) according to the manufacturer's instructions. RNA was eluted in RNase-free water and analyzed for purity (260/280 nm ratios of approximately 2.0 were considered acceptable) and nucleotide content on a spectrophotometer.

Equal amounts of RNA (150-250 ng from whole DRG, 500 ng from cell cultures) were reversed-transcribed using M-MLV (200 U/rxn), dNTPs and oligo (dT) 12-18 primers (Invitrogen) in a 20 μL reaction volume according to the manufacturer's instructions. PCR reactions (25 μL) using Go Taq Flexi DNA polymerase (Promega) were then performed from cDNA on a thermal cycler (MJ Mini; BioRad). The PCR conditions were 95° C. (2 min), cycled 35 times from 95° C. (30 sec) to 60° C. (30 sec) to 72° C. (30 sec), 72° C. (5 min), 4° C. Reactions performed without cDNA served as negative control. PCR products were run on an agarose (1.5%) gel containing a DNA staining reagent (SafePinky; GenDepot) and visualized on a UV transilluminator.

### 2-24. Quantitative Real-Time PCR

Gene expression was performed on cDNA from DRG using a Power SYBR Green PCR Master Mix (Applied Biosystems) and pairs of target-specific primers (500 nm) in MicroAmp optical tubes (20 μL reaction volume) on a 7500 Real-Time PCR system (Applied Biosystems). The PCR conditions were 50° C. (2 min), 95° C. (10 min), and cycled 40 times at 95° C. (15 sec) to 60° C. (1 min). Samples were run in triplicate. Data were analyzed using the built-in 7500 software (v2.0.4; Life Technologies) and expression was determined relative to a reference gene (Gapdh) in adult DRG for cultures or contralateral DRG for nerve injury experiments using the comparative Ct method (Schmittgen and Livak, 2008). Primers were designed using Primer-BLAST software (NIH) (Ye et al., 2012). Primers were selected based on specificity to the desired target gene upon BLAST search, overlap of the exon-exon boundary, lack of potential hairpin-forming or self-priming regions, single peak in the dissociation curve (single band PCR product) and equivalent amplification efficiency (linear shift in Ct value upon serial dilution of cDNA). Products of reverse transcription reactions omitting RNA or M-MLV (-RT) served as negative controls.

### 2-25. Laser Scanning Confocal Imaging and Analysis

For analysis of neurite density in microfluidic co-cultures, single z-section low magnification (×10) confocal images at 0.5 zoom (LSM700, Zeiss) were acquired of β-tubulin III immunofluorescence (647 nm emission) along the full length of the neurite compartment. Gamma high channel images were exported as an unaltered TIFF using Zeiss imaging software (v8.1; ZEN 2012 SP1, Zeiss). Images were converted to black/white by adjusting to a set threshold, scale calibrated and horizontal pixel density was measured using the Plot Profile function in Image J (v1.46r, NIH). The distance to 50% neurite density was calculated from the normalized cumulative pixel density. 6-8 images per microfluidic device were acquired, n=3 devices per group were used. For analysis of neurite fragmentation in NK-DRG co-cultures, 5-10 fields of view were randomly selected from each coverslip in a non-systematic manner for acquisition in single or double z-section confocal images at low magnification (×20) and 0.5 zoom (LSM700, Zeiss). For total neurite fragmentation gamma, high channel images of β-tubulin III immunofluorescence were exported as an unaltered TIFF. In Image J, images were scale calibrated (1.6 pixels/μm) and brightness threshold set in black/white mode, creating a whole neuron silhouette for selection. Neurite fragments were selected using the particle analysis function (size 0.5-25 μm², circularity 0-1) and saved as a drawing. The total area and particular area values (μm2) were obtained using the Measure function and used to calculate the percent neurite fragmentation for each field of view. For analysis of β-tubulin III and STMN2 labeling in sciatic nerve tissue, a composite of the full-length nerve was created from individual z-section images acquired at ×10 magnification and 0.5 zoom along the length of the nerve (LSM700; Zeiss). Images containing crush site, as well as proximal and distal regions (±1 mm from the crush site) were exported to ImageJ, scale calibrated (0.8 pixels/μm) and brightness threshold set in black/white mode. The mean pixel density from a 9,000 μm² selection of the nerve mid-image was recorded from 3-4 images per nerve region, per mouse, per treatment. Co-localized regions of RAE1 double-labeling with β-tubulin III and STMN2 were exported using the co-localization function on the Zen 2012 software after setting for threshold image intensity in both fluorescence channels (1,000 units per channel).

### 2-26. Statistical Analysis

Comparisons between two groups of data were made with Student's t test (paired or unpaired) or Mann-Whitney U test for non-normal distributions (confirmed with Kolmogorov-Smirnov test). Comparisons between three or more groups of data were made with One-way ANOVA. Two-way ANOVA tests were used to compare the effects of treatments on neuronal cytotoxicity and behavioral sensitivity assays. Data are presented as mean±standard error of the mean unless otherwise stated. Analysis were performed using GraphPad Prism version 5.00 for Windows, GraphPad Software, San Diego California USA, www.graphpad.com. No statistical methods were used to predetermine sample sizes, which were based on previous literature and availability of animals. For behavior tests, experimenters were blinded either to the treatment or genotype of the mice during both surgery and sensory testing. Treatments were assigned to litter mates at random by an independent observer. p<0.05 was considered significant.

### Example 1. Confirming that Activated Mouse NK Cells Induce Cytotoxicity in Mouse Embryonic Sensory Neurons by an RAE1-Mediated Mechanism

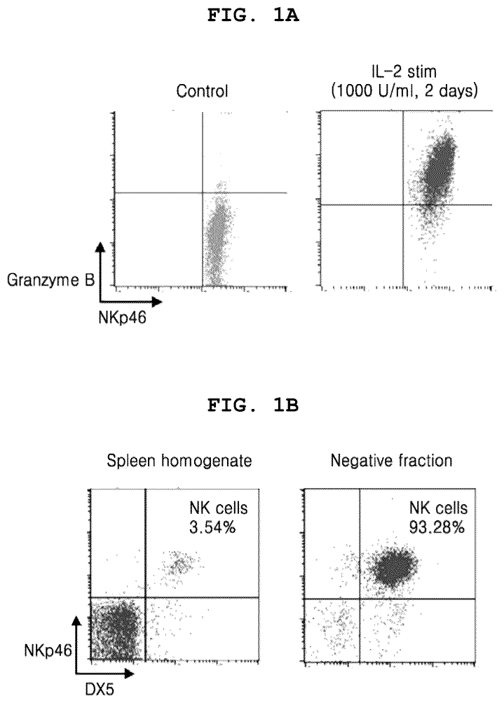

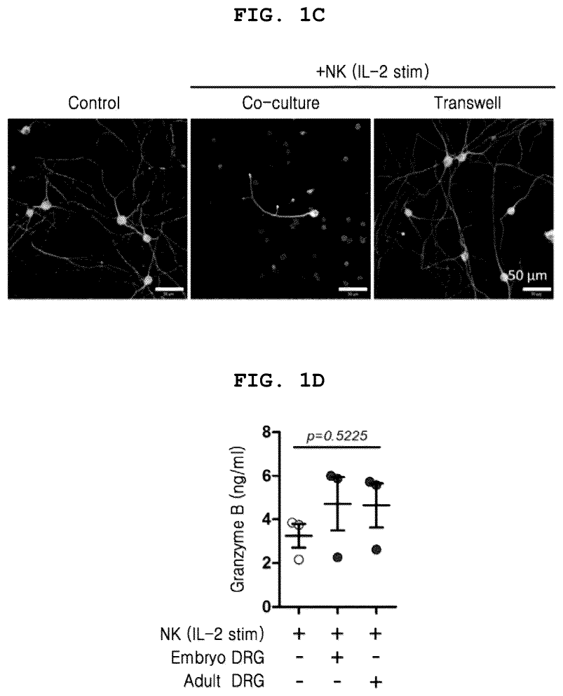

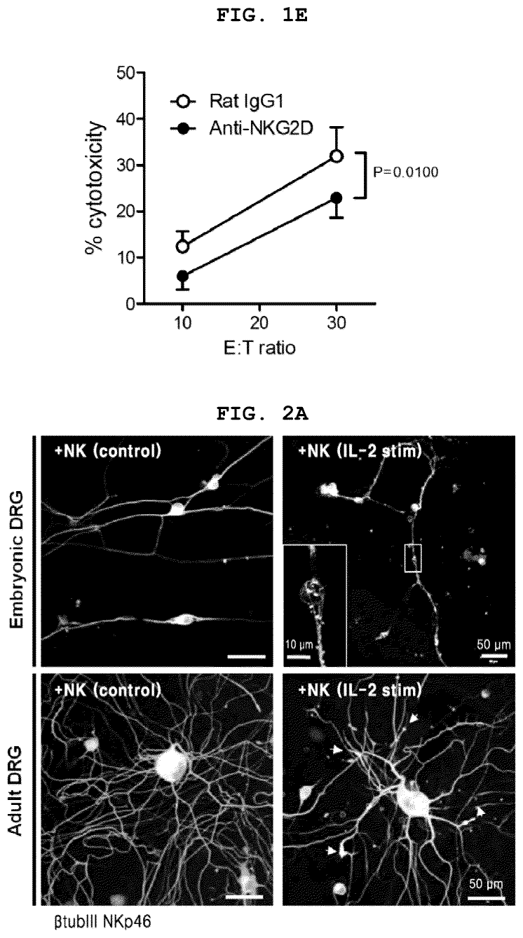

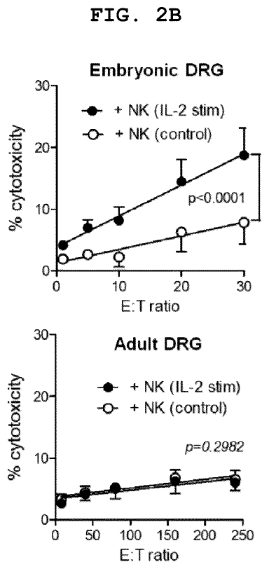

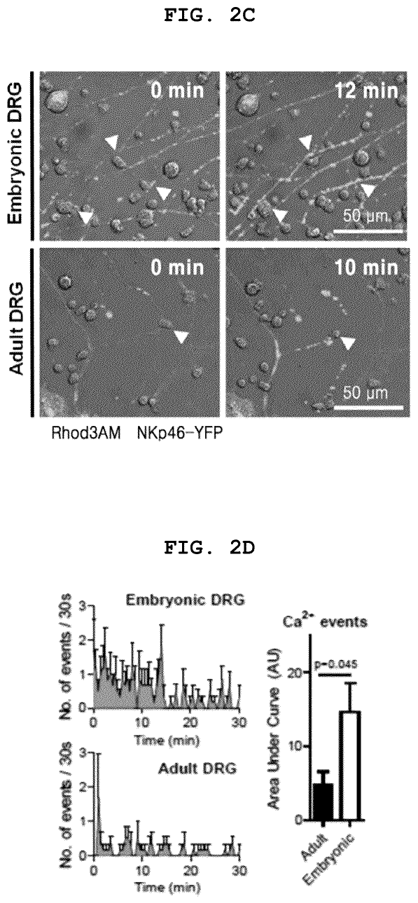

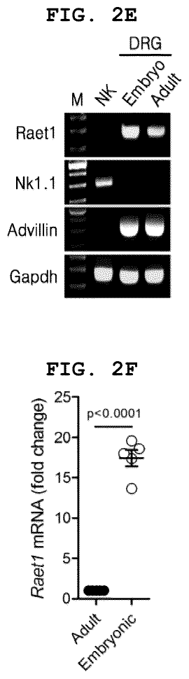

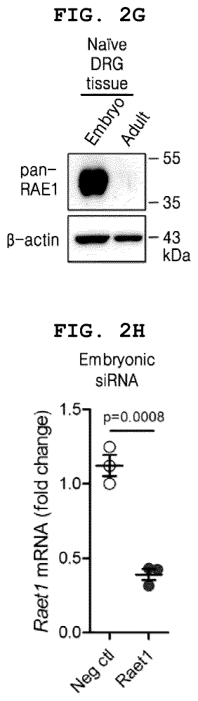

Mouse NK cells are activated by cytokine IL-2, which induces cytotoxic attack by increasing intracellular content of granzyme B (FIG. 1A). In this Examples, the effects of IL-2-stimulated mouse NK cells on DRG neurons acutely isolated from embryonic (E15) and adult mice (<24 h in vitro) were examined using isolated, unstimulated splenic NK cells as a control (FIG. 1B). As reported, mouse embryonic DRG neurons were highly susceptible to mouse NK cell-mediated cytotoxicity (FIGS. 2A and 2B). Separating mouse NK cells and embryonic DRG with a trans-well membrane prevented neurite fragmentation (FIG. 1C) despite the presence of nanogram amounts of granzyme B in the culture media (FIG. 1D), indicating that the cytotoxic effect on mouse DRG neurons requires direct contact with mouse NK cells. In contrast, in acutely isolated adult DRG neurons co-cultured with IL-2-stimulated NK cells, no evidence of cell lysis by LDH release cytotoxicity assay was found even at much higher effector-to-target (E:T) ratios (FIG. 2B), although some neurites appeared truncated (FIG. 2A). Time-lapse confocal imaging of Dil-labelled DRG neurons co-cultured with IL-2 stimulated yellow fluorescent protein (YFP)-expressing NK cells revealed that stimulated NK cells were highly motile, enabling direct cell-cell contacts between NK cells and sensory neurons. In embryonic DRG, this led to neurite destruction and cell death (not shown 1); however, acutely cultured adult DRG neurite membranes remained largely intact with no attendant cell death despite similar direct cell-cell contacts (not shown 2). Next, in the present Example, mouse DRG neurons were loaded with the Ca²⁺-sensitive indicator rhodamine 3-AM before applying IL-2 stimulated mouse NK cells, and the time-lapse imaging was performed. NK cell contacts were often synchronous with intracellular Ca²⁺ events in embryonic DRG neurites (FIGS. 2C, 2D, and not shown 3), leading to neurite fragmentation. While Ca²⁺ events were also observed in adult DRG after NK cell contact (FIGS. 2C and 2D), and occasionally, led to neurite fragmentation (not shown 4), they occurred at a much lower frequency than in embryonic neurons (FIG. 1D). The protein Retinoic Acid Early 1 (RAE1), encoded by the Raet1 gene family (α, β, γ, δ, ε), acts as a membrane-bound ligand for the mouse activating receptor NKG2D (Cerwenka et al., 2000), which has previously been implicated in NK cell-mediated lysis of embryonic DRG (Backstrom et al., 2003); however, a functional requirement for Raet1 in mouse NK cytotoxicity against mouse DRG neurons has yet to be shown. First, in this Example, it was confirmed that NK cytotoxicity against embryonic DRG neurons may be attenuated by an NKG2D receptor blocking antibody (FIG. 1D). Using RT-PCR with universal primers designed to detect all five Raet1 isoforms (α, β, γ, δ, ε), Raet1 transcripts were observed in acutely dissociated embryonic and adult DRG neurons (FIG. 2E). Quantitative RT-PCR (qRT-PCR) showed that Raet1 transcripts were 17 times more abundant in embryonic DRG relative to adult DRG (FIG. 2F). This difference was reflected at the protein level: a single large band at approximately 40 to 50 kDa was detected by a pan-RAE1 antibody in western blot of embryonic but not adult DRG tissue (FIG. 2G). To assess the functional contribution of Raet1 in embryonic DRG neurons, all Raet1 isoforms were selectively knocked out using Raet1 siRNA (FIG. 2H). Compared to negative control siRNA, transfection of embryonic DRG neurons with Raet1-selective siRNA led to a 20% reduction in NK cell-mediated lysis (FIG. 2I). These results show that Raet1 expression in embryonic mouse DRG neurons is involved in the cytotoxicity induced by direct engagement with stimulated mouse NK cells.

### Example 2. Confirming that Nerve Injury Drives RAE1 Expression in Adult Sensory Neurons Allowing Cytotoxic Attack by Activated NK Cells

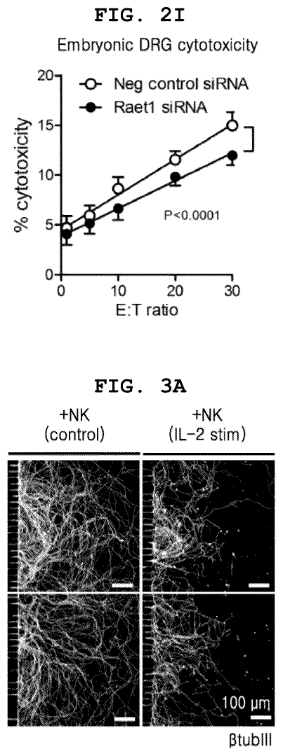

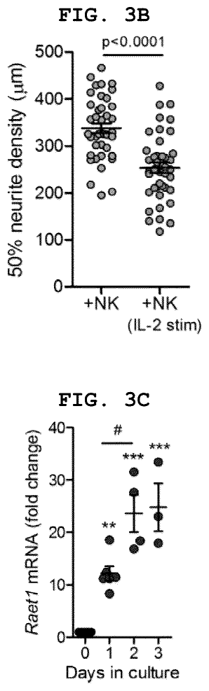

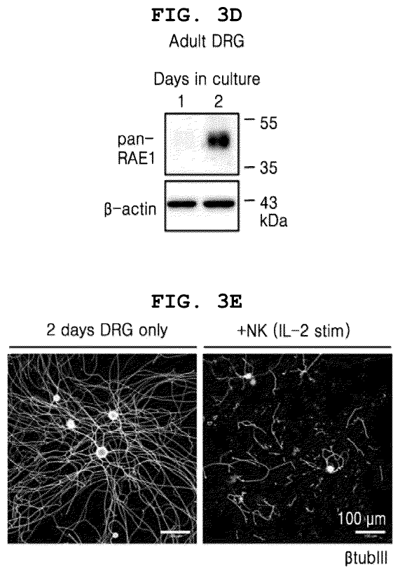

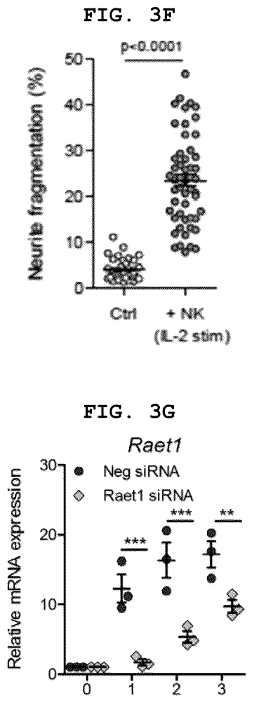

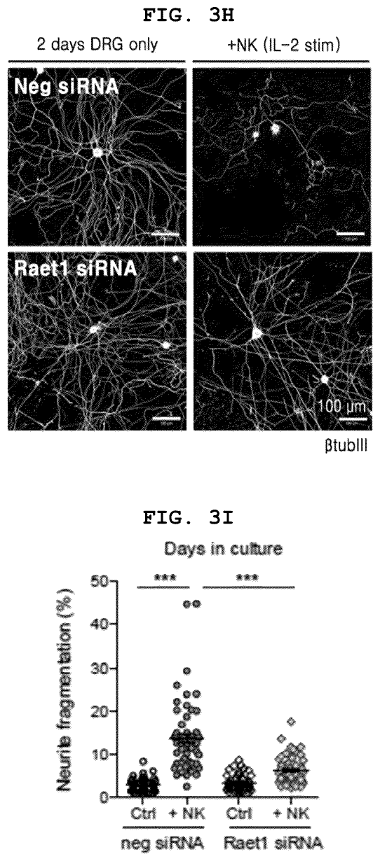

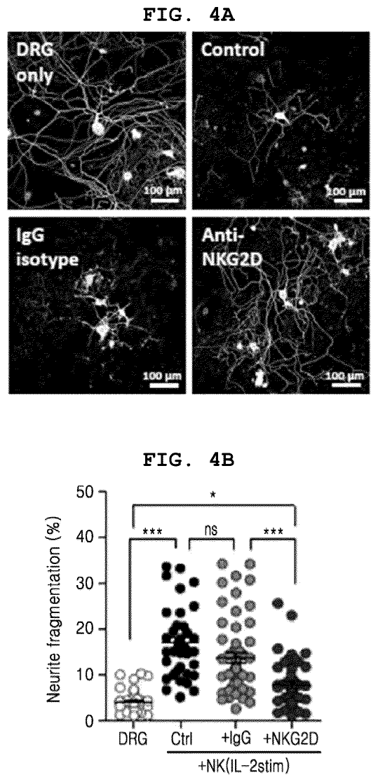

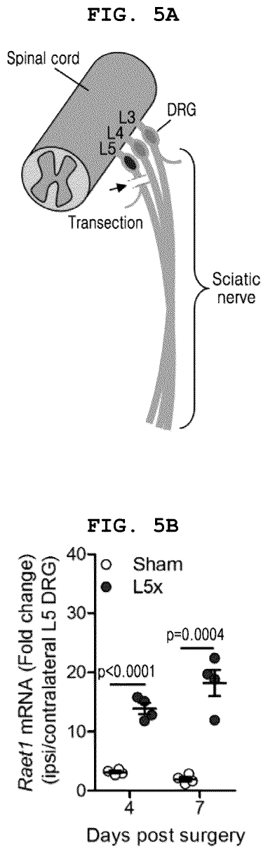

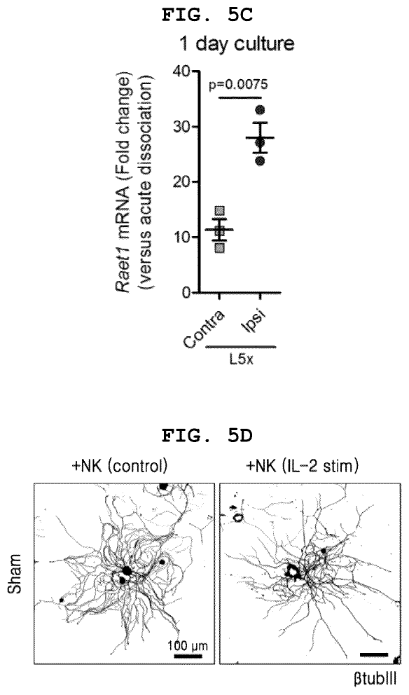

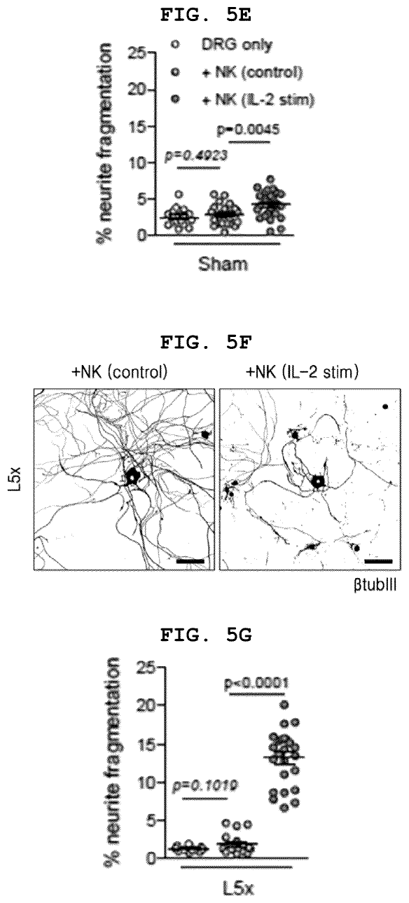

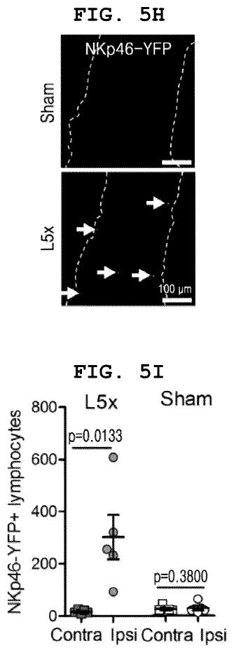

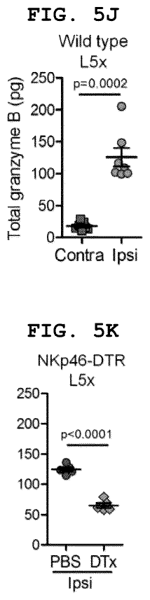

To investigate the potential effects of activated mouse NK cells on adult sensory neurons, mouse DRG neurons were cultured in a microfluidic chamber (5 days in vitro), which allows the selective exposure of their axons to stimulated mouse NK cells in the neurite compartment. Following exposure to stimulated NK cells, 25% loss of neurite coverage relative to isolated control NK cells was observed (FIGS. 3A and 3B). Next, it was examined whether RAE1 plays a role in NK cell-mediated neurite fragmentation of these longer-term adult DRG cultures. Raet1 mRNA was time-dependently upregulated in adult DRG cultures (FIG. 3C), with corresponding de novo expression of RAE1 protein after 2 days in vitro (FIG. 3D). Consistent with a role for RAE1 in susceptibility to NK cell attack, adult DRG neurons cultured for 2 days displayed over a 10-fold increase relative to controls in neurite fragmentation in the presence of stimulated NK cells (FIGS. 3E and 3F). Transfection of dissociated adult DRG neurons with Raet1 siRNA prior to culture delayed Raet1 upregulation (FIG. 3G) and reduced the ability of stimulated NK cells to fragment DRG neurites relative to negative control siRNA (FIGS. 3H and 3I). It was also confirmed that fragmentation of adult DRG neurites (2 days in vitro) can be reduced by blocking the NKG2D receptor on NK cells prior to co-culture (FIGS. 4A and 4B). Thus, de novo expression of RAE1 in long-term adult DRG cultures confers susceptibility to NKG2D-mediated neurite fragmentation by NK cells. It was also examined whether Raet1 upregulation in dissociated DRG in vitro occurs in injured cells in vivo. To address this, the fifth lumbar spinal nerve distal to the DRG (L5x) in adult mice was cut (FIG. 5A). An injury-dependent upregulation of Raet1 was revealed by qRT-PCR using RNA isolated from L5 DRG at 4 and 7 days after L5x injury (FIG. 5B). The difference in Raet1 transcript levels between ipsilateral and contralateral L5 DRG 7 days after L5x injury was maintained over the first 24 h in culture (FIG. 5C). When IL-2 stimulated mouse NK cells were introduced to these cultures, mouse neurons from injured DRG (L5x) exhibited 3.5-fold more fragmentation of neurites (FIGS. 5F and 5G) than those from DRG from uninjured (sham) animals (FIGS. 5D and 5E). These results suggest that peripheral nerve injury drives Raet1 induction in adult sensory neurons and this expression correlates with NK cell-mediated neurite destruction.

### Example 3. Confirmed that Stimulated NK Cells Degenerate Partially Injured Sensory Axons

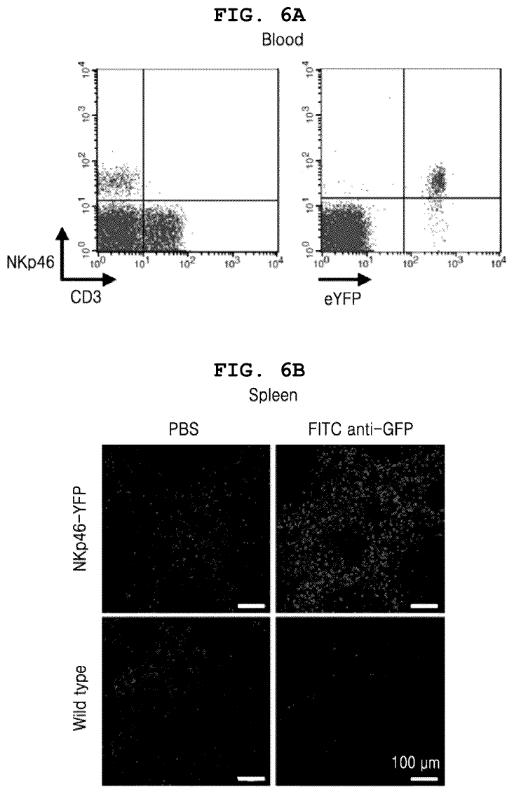

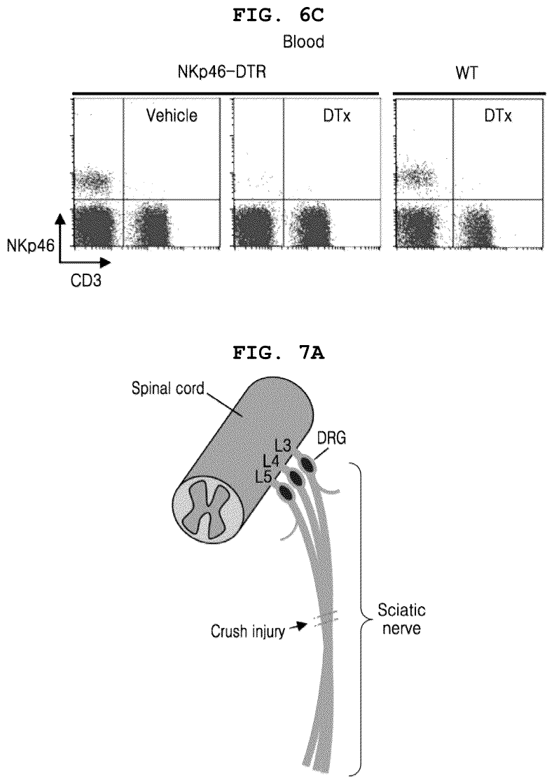

To characterize the response and function of mouse NK cells in vivo, a mouse expressing the inducible Cre recombinase (iCre) driven by the Ncr1 gene promoter (Ncr1^{icre}) (Narni-Mancinelli et al., 2011) was used to cross with reporter mice which express either enhanced yellow fluorescent protein (Rosa26^{eyfp}; YFP) (Srinivas et al., 2001) or the diphtheria toxin receptor (Rosa26^{dtr}; DTR) (Buch et al., 2005) following cre-mediated recombination. Ncr1 encodes the NKp46 receptor, which is expressed on all NK cells, as well as tissue-resident ILCs, including group 1 ILCs (ILC1s) and a subset of group 3 ILCs (NCR¹⁺ ILC3s). This genetic approach allowed to identify (via YFP expression) or systemically deplete (via DTR) NKp46⁺ cells in vivo (FIGS. 6A, 6B and 6C). YFP-positive NK cells were not observed in sciatic nerve in naive mice or sham surgery mice (FIGS. 5H and 5I). In contrast, spinal nerve transection (L5x) led to the marked recruitment of YFP-positive NK cells to the injured sciatic nerve (FIGS. 5H and 5I). NK cell infiltration to the sciatic nerve was paralleled by a large increase in granzyme B levels at 7 days post-injury (FIG. 5J), which was significantly reduced by prior systemic depletion of NK cells by DTx treatment in NKp46-DTR mice (FIG. 5K).

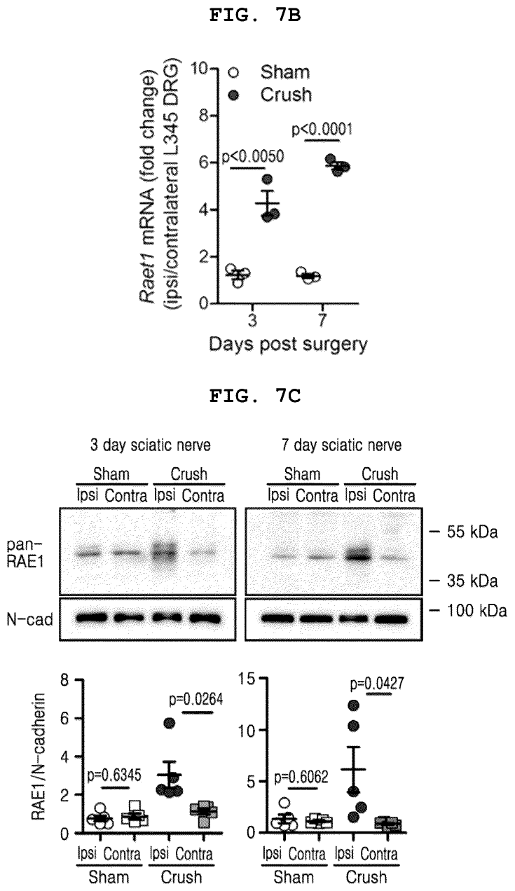

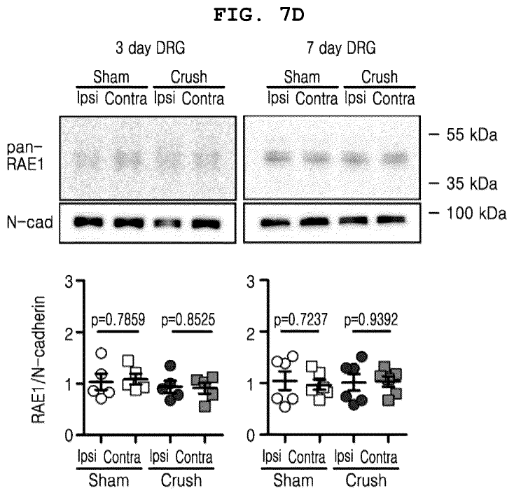

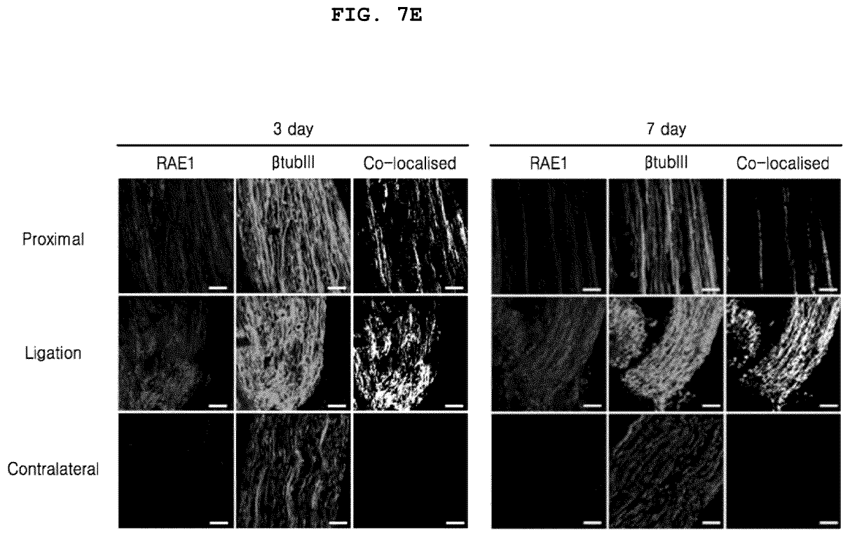

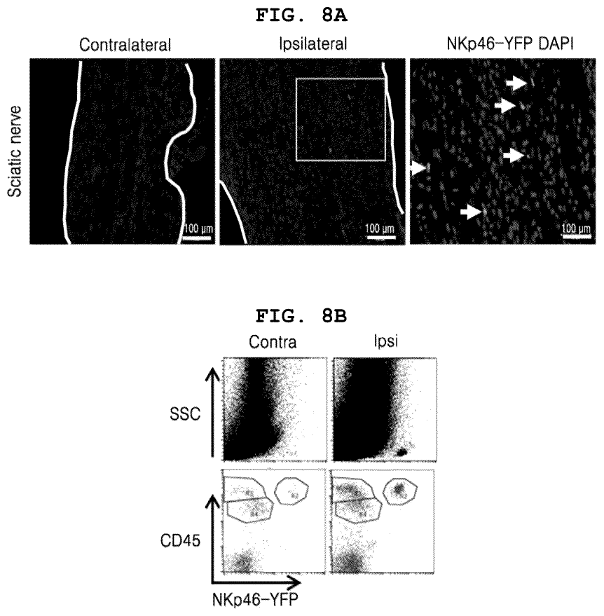

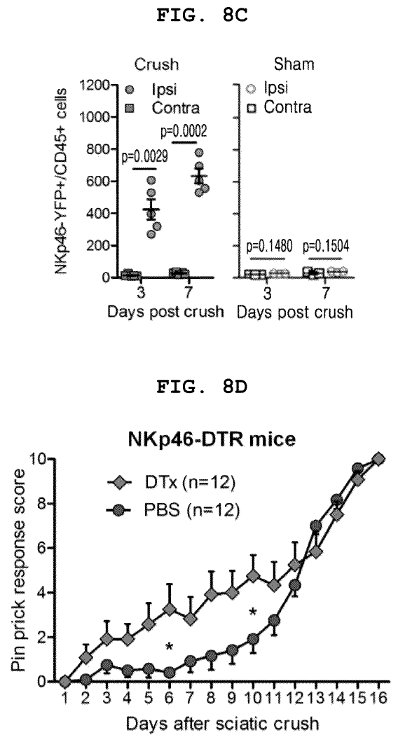

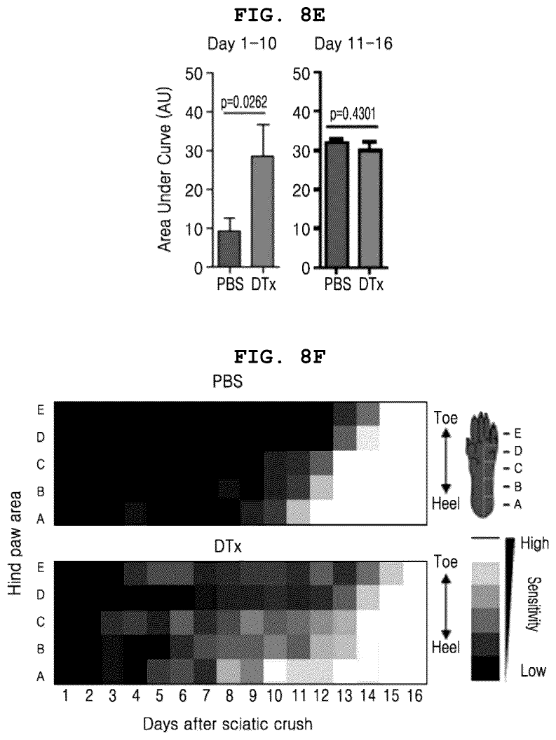

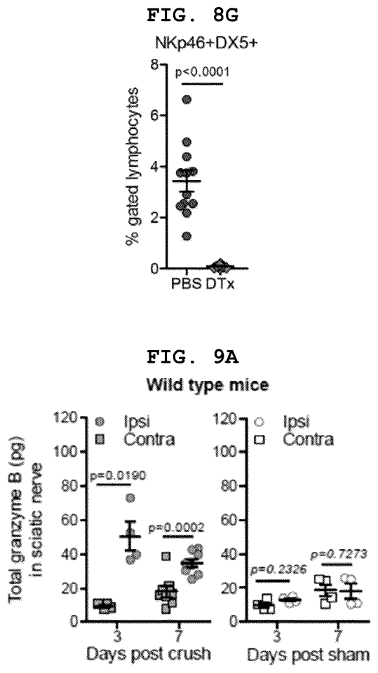

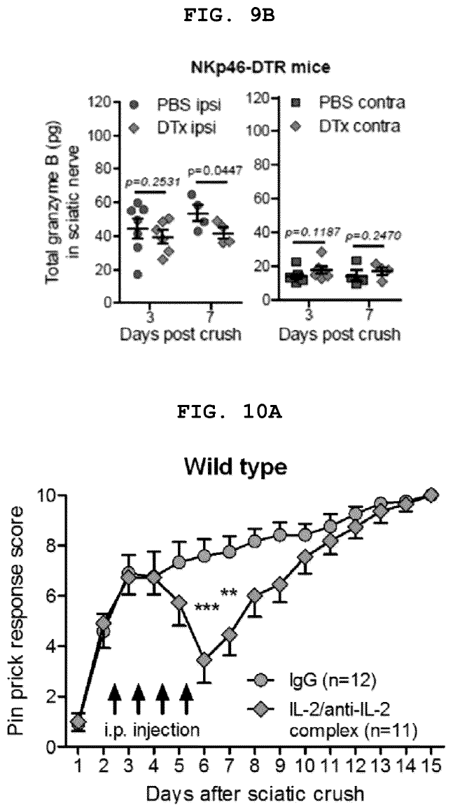

To examine the potential role of NK cells in the degeneration of axons after injury, complete sciatic nerve crush injury was performed with fine forceps in mice (FIG. 7A). Consistent with the effect of nerve transection, crush injury drove Raet1 expression in mouse ipsilateral DRG (FIG. 7B). As RAE1 is known to be controlled by various forms of post-transcriptional modification (Raulet et al., 2013), Western blot of lysates of sciatic nerve and DRG tissue following crush and sham injury was performed using a pan-RAE1 antibody, which recognizes all isoforms of the protein. Crush injury induced a higher molecular weight band (approximately 45 kDa) in ipsilateral sciatic nerve but not in contralateral nerve or sciatic nerve from sham-injured mice. The 45 kDa size indicates an increase in the glycosylated mature form of RAE1 (Arapovic et al., 2009) in ipsilateral injured sciatic nerve (FIG. 7C) but not in the DRG (FIG. 7D). Furthermore, chronic ligation of the mouse nerve to block axonal transport revealed RAE1 immunolabeling within axons proximal to the crush site (FIG. 7E). These data suggest that there is axonal-specific shipping and/or translation of RAE1 protein at the injury site. Crush injury in NKp46-YFP mice also caused recruitment of NK cells within damaged ipsilateral sciatic nerve (FIG. 8A), and flow cytometry showed that the number of CD45⁺YFP⁺ lymphocyte-gated events (indicative of NK cells) detected in ipsilateral sciatic nerve (FIG. 8B) increased by 30-fold 3 days after injury and a further 50% by day 7 (FIG. 8C). Crush injury also raised levels of granzyme B in the sciatic nerve (FIGS. 9A and 9B). Using two-photon imaging of the sciatic nerve in vivo 3 days after crush injury, NKp46-YFP cells rolling along the wall of intra-neural blood vessels (not shown 5) was observed. Within the sciatic nerve itself, NK cells were highly motile and displayed multipolar morphology, reminiscent of stimulated NK cells in vitro (not shown 6). These cells are ideally placed to interact functionally with damaged mouse peripheral nerve axons that selectively express the NK cell target protein RAE1. No YFP⁺ cells were observed by two-photon imaging in the nerves of sham injured mice (Data not shown).

### Example 4. Confirming that Endogenous Mouse NK Response Attenuates Post-Injury Sensitivity

Sciatic nerve crush injury induces acute loss of sensation and rapid Wallerian degeneration of primary afferent axons distal to the injury site, followed by sensory recovery within approximately two weeks (Ma et al., 2011). After crush with fine forceps, complete loss of response to pin-prick stimulation of the lateral hind paw was confirmed in all mice (FIGS. 8D and 8E). Control (PBS-treated) NKp46-DTR mice showed a general pattern of sensory recovery (Ma et al., 2011; Painter et al., 2014), remaining almost completely insensitive until a relatively rapid recovery beginning around 10 to 11 days post injury (FIG. 8D, PBS). To investigate the role of NK cells in axonal integrity following nerve crush injury in vivo, peripheral sensitivity was assayed in NKp46-DTR mice chronically depleted of NK cells by diphtheria toxin (DTx) treatment (FIG. 8G). These mice displayed an early and persistent sensitivity to pin prick stimulation (FIG. 8D, DTx). In contrast to control mice, which showed a heel-to-toe pattern of sensory recovery (FIG. 8F, PBS), NK cell-depleted mice showed an anatomically broad pattern of responses to stimulation throughout the hind paw prior to full recovery (FIG. 8F, DTx), indicative of remaining sporadic innervation throughout the tissue.

### Example 5. Confirming that Stimulated Mouse NK Cells Degenerate Partially Injured Sensory Axons in Traumatic Peripheral Neuropathy Mouse Model

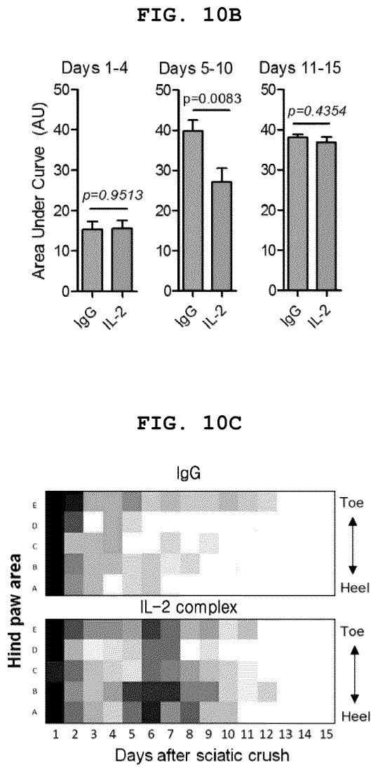

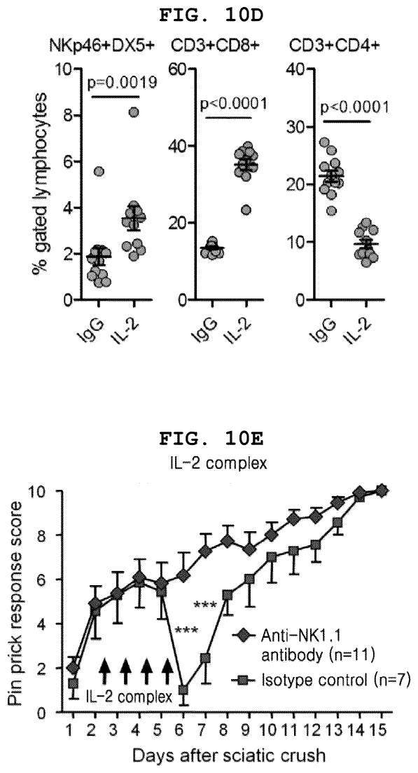

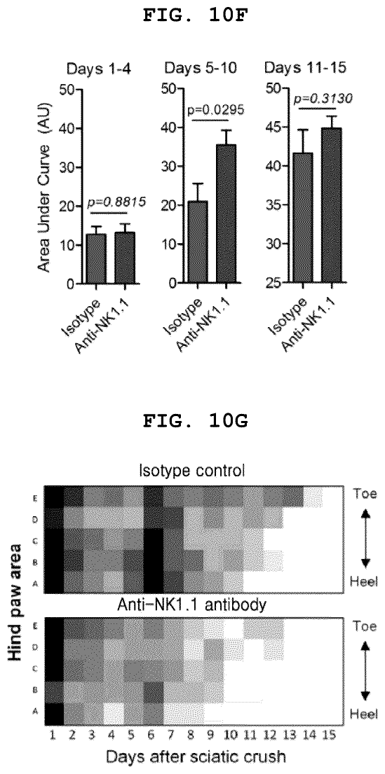

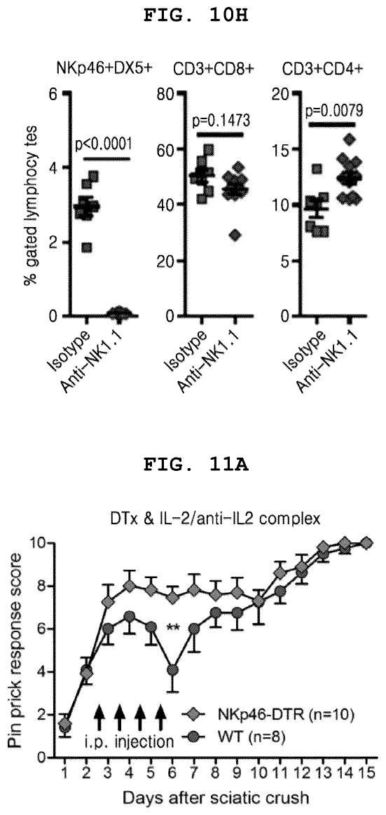

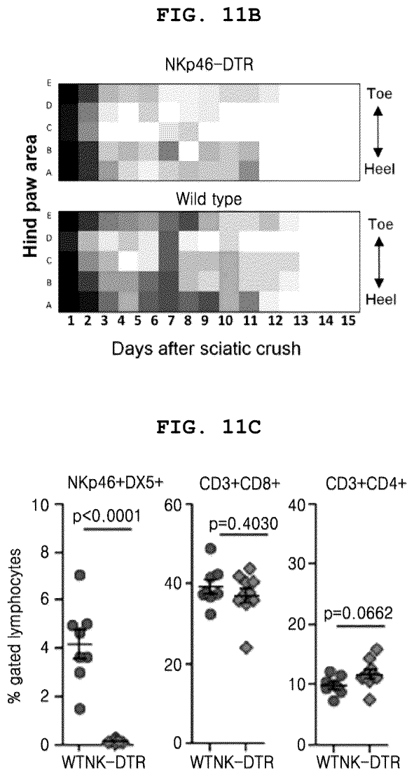

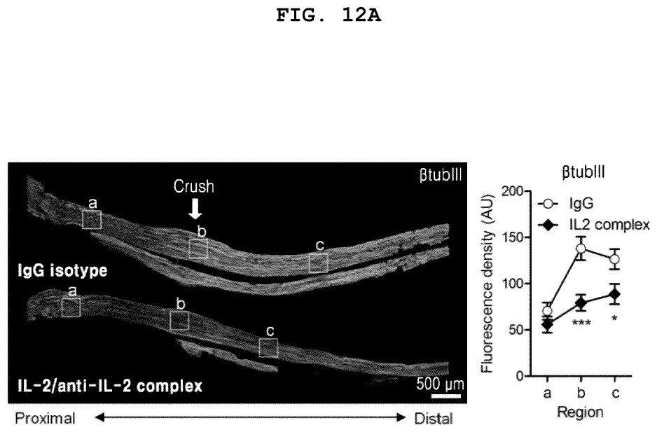

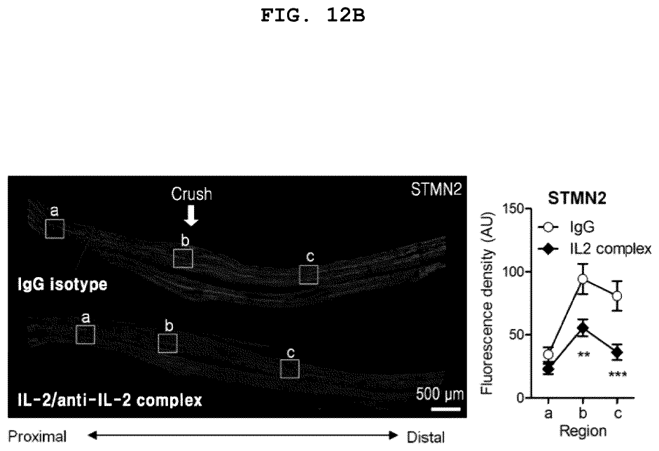

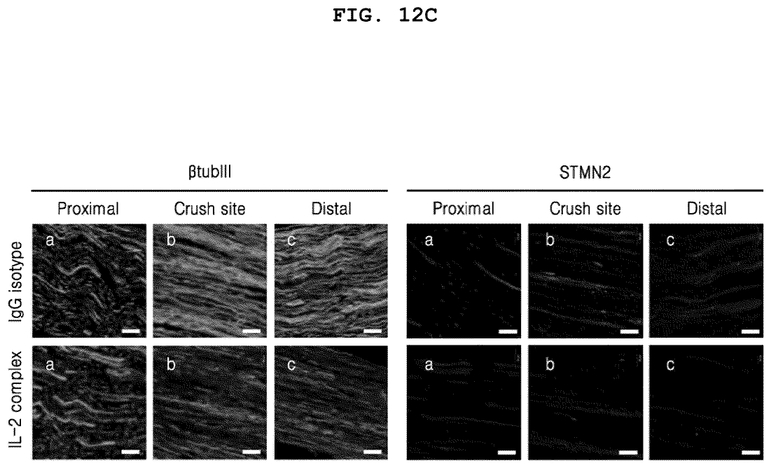

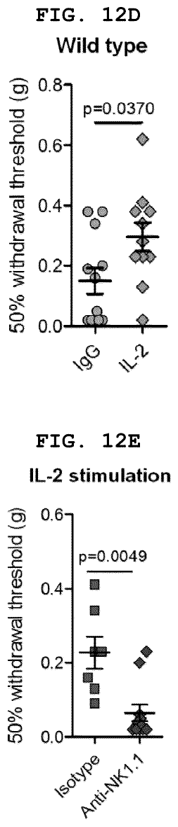

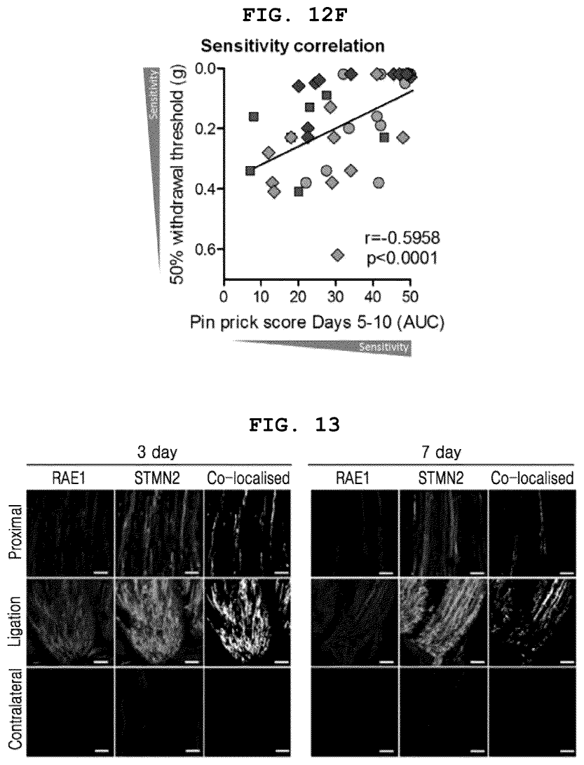

To determine whether NK cells are able to selectively degenerate partially injured axons, a consistent partial sciatic crush was delivered by using a fine hemostat with 30 μm spacer. Control animals generally showed some residual pin prick sensitivity one day after crush, followed by a rapid functional recovery to over 60% of maximum by day 6 (FIGS. 10A, IgG), suggesting that a proportion of fibers were incompletely axotomized. This partial sensitivity was maintained until 11 days post-crush, at which point sensory recovery occurred in a heel-to-toe pattern, reaching full recovery by day 15 (FIG. 10B, IgG), consistent with regenerating fibers. To replicate the stimulation of NK cells by IL-2 in vivo, mice were systemically treated with an IL-2/anti-IL-2 monoclonal (S4B6) antibody complex. IL-2/anti-IL-2 antibody complex treatment is known to enhance both NK and CD8⁺ T cell populations (Boyman et al., 2006). Indeed, both NKp46⁺DX5⁺ and CD3⁺CD8⁺ (FIG. 10D) cell populations were enriched in the blood of IL-2 complex-treated mice at the end of the experiment (Day 16) compared to controls, whereas the proportion of CD3⁺CD4⁺ T cells was decreased (FIG. 10D). Compared to IgG-treated controls, animals treated with the IL-2 complex after injury showed a significant acute deterioration in pin prick response (FIGS. 10A and 10C, IL-2 complex); however, both groups of animals regained full pin prick sensitivity at around two weeks (FIG. 10B). Further, the specific contribution of NK cells in this process was determined. The effect of IL-2 complex was examined in the mice, after pre-treatment with an anti-NK1.1 antibody to systemically deplete NK cells. In isotype control-treated mice, IL-2 complex treatment again resulted in acute loss of the pin prick response (FIGS. 10E, 10F and 10G, Isotype), but NK cell-depleted mice maintained a constant level of ‘retained’ sensitivity throughout the paw until regeneration continued from 11 days after injury (FIGS. 10E, 10F, and 10G). The dependence of NK cells on the effect of IL-2 complex was further confirmed in DTx-treated wild-type and NKp46-DTR mice (FIGS. 11A and 11B). Flow analysis of peripheral blood after recovery (Day 16) confirmed depletion of NKp46⁺DX5⁺NK cells by anti-NK1.1 in wild-type mice (FIG. 10H) and by DTx in NKp46-DTR (FIG. 11C). No significant difference was observed in CD3⁺CD8⁺ T cell populations, although the proportion of CD3⁺CD4⁺ T cells was 20% higher in the antibody depleted group compared to isotype controls (FIG. 10H). To determine the mechanism behind the NK cell-dependent sensory loss after IL-2 complex treatment, axon fibers within the sciatic nerve were examined 6 days after partial crush. The increase in density of β-tubulin III-labelled axon fibers from the crush site extending distal throughout the sciatic nerve in control (IgG-treated) mice was reduced by IL-2 complex-treatment (FIGS. 12A and 12C, insets B and C). Axon density in the proximal nerve region, however, was not different between treatment groups (FIGS. 12A and 12C, inset A). The distribution of the microtubule-associated protein stathmin 2 (STMN2), which is specifically expressed by injured DRG neurons, was also examined (Cho et al., 2013). Similar to the distribution of β-tubulin III, STMN2 labeling was observed throughout the crushed nerve, and was reduced by IL-2 complex treatment at the crush site and distal regions relative to IgG controls (FIGS. 12B and 12C, insets B and C). STMN2 also co-localized with RAE1 immunolabeling at the injury site (FIG. 13). These results suggest that the temporary sensory loss observed with IL-2 complex treatment after partial crush injury is due to removal of injured axon fibers within the sciatic nerve.

### Example 6. Confirming that Clearance of Partially Damaged Axons Alleviates Pain Hypersensitivity after Injury in Traumatic Peripheral Neuropathy Mouse Model

In this Example, the mechanical threshold after recovery from partial crush injury (Days 15-16) was assessed by measuring responses to von Frey hair stimulation of the lateral hind paw. IL-2 complex treatment resulted in significantly higher mechanical thresholds in the hind paw of the previously crushed (ipsilateral) limb, compared to control (IgG) animals, which displayed relative hypersensitivity (FIG. 12D). Furthermore, normalization of mechanical thresholds by IL-2 complex treatment after partial crush was prevented by prior depletion of NK cells (FIG. 12E). Combining data from all animals revealed that the cumulative pin prick sensitivity (area under the curve) during the peak effect of treatment (days 5 to 10) correlates with the resulting mechanical sensitivity of the injured limb after recovery from a partial crush (FIG. 12F). Taken together, these data suggest that effective clearance of partially damaged mouse axons by mouse NK cells following nerve injury is likely to be important to prevent the appearance of persistent mechanical sensitivity from retained partially damaged axons within the sciatic nerve.

### Example 7. Confirming that NK Cells have Function to Remove Nerves in Mouse Model of Peripheral Neuropathy Caused by Administration of Anticancer Drug

To examine whether NK cells are also able to selectively remove injured or abnormal nerves in peripheral neuropathy caused by administration of an anticancer drug, interactions between NK cells and anticancer drug-exposed sensory nerve cells were observed and ligand expression of NK cells was measured in mouse models of peripheral neuropathy caused by administration of the anticancer drug.

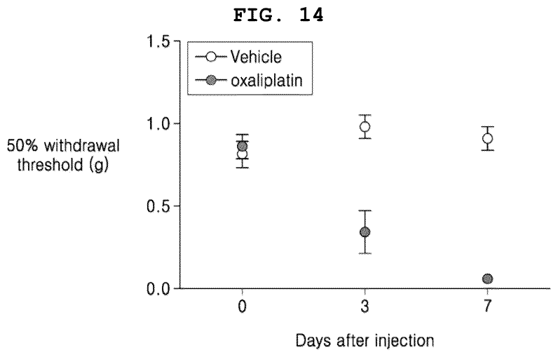

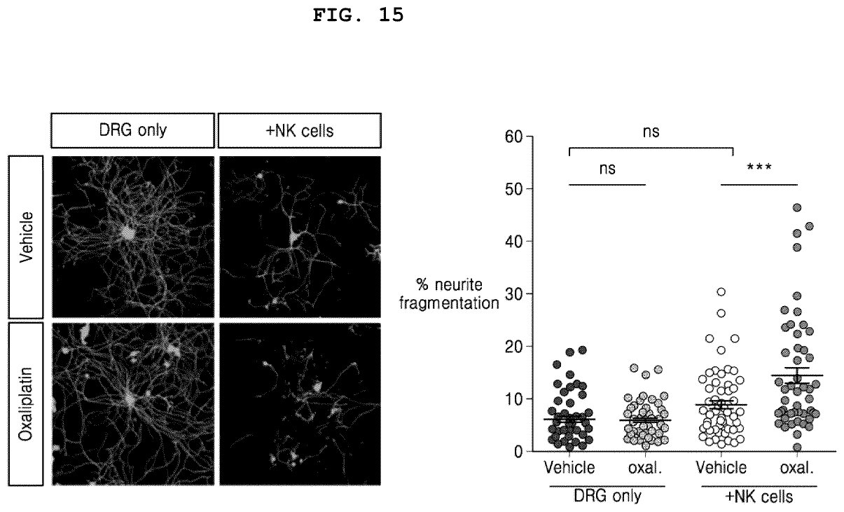

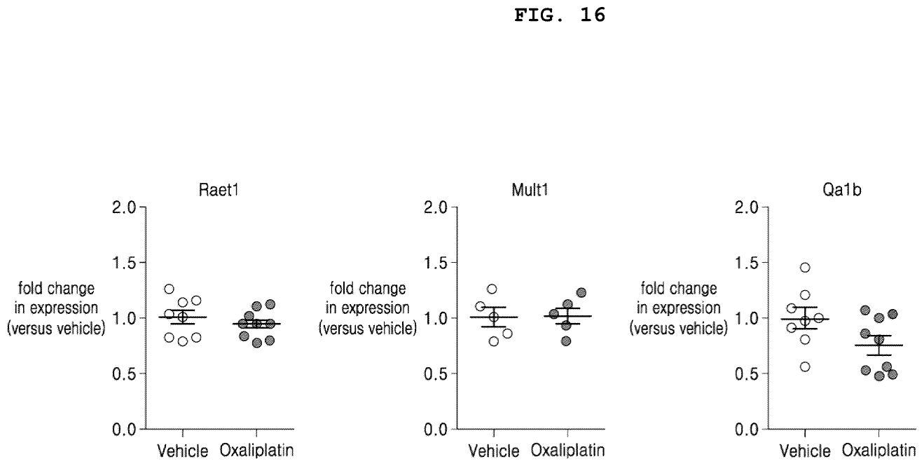

In detail, an anticancer drug (oxaliplatin, 10 mg/kg) was injected into the intraperitoneal cavity of mice to induce peripheral neuropathy due to administration of the anticancer drug, and the injured mouse peripheral sensory nerve cells were isolated on day 7, primarily cultured, and then co-cultured with mouse NK cells to examine the interaction between the two cells. As a control, peripheral sensory nerve cells of a normal mouse were used. FIG. 14 shows results confirming that peripheral neuropathy caused by administration of the anticancer drug was induced since pain was induced on day 7 in the anticancer drug-administered group. FIG. 15 shows results of examining the interaction between NK cells and peripheral sensory nerve cells which were cultured on day 7 after administration of the anticancer drug. FIG. 16 shows results of examining ligand expression of NK cells in sensory nerve cell tissues on day 7 after administration of the anticancer drug.

As a result, as shown in FIG. 15, it was confirmed that mouse NK cells selectively removed the anticancer drug-exposed sensory nerve cells in mice.

Further, as shown in FIG. 16, no changes in NK cell-activating ligands Raet1 and Mult1 were observed, but an inhibitory ligand Qa1b was significantly reduced, indicating that attack of NK cells was induced due to reduction of the inhibitory ligand in the target cells.

These results suggest that NK cells can also be used to remove injured nerves in peripheral neuropathy caused by administration of the anticancer drug, which may be mediated by Qa1b.

### Example 8. Confirming that Infiltration of NK Cells into Injured Nerve Site Increases in Mouse Models with Central Nervous System Excitotoxicity

To examine whether NK cells are able to selectively remove injured or abnormal nerves in the central nervous system, infiltration of NK cells into the central nervous system was examined in central nervous system excitotoxicity-induced mice.

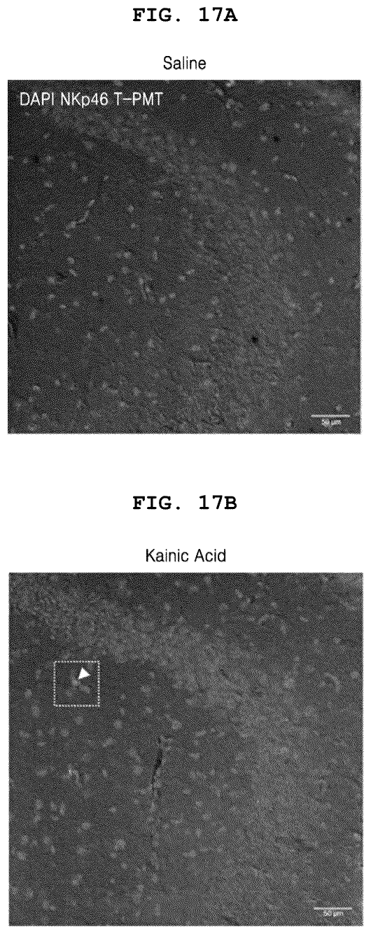

In detail, Kainic acid (35 mg/kg) was injected into the abdominal cavity of mice to induce excitotoxicity of the central nervous system, and saline-injected mice were used as a control. Central nervous system excitotoxicity is a phenomenon observed in representative brain diseases such as Alzheimer's disease, epilepsy, Parkinson's disease, etc. (Dong et al., 2009), and a central nervous system excitotoxicity model is also known as a representative seizure model. Kainic acid is a potent neuroexcitatory amino acid agonist that acts by activating the glutamate receptor, which is a major excitatory neurotransmitter in the central nervous system, and treatment of mice with high concentrations of kainic acid leads to excitotoxicity and neurodegeneration in the central nervous system (Levesque et al., 2013). FIGS. 17A to 17C show images of selective infiltration of NK cells into brain tissues in which excitotoxicity of the central nervous system was induced.

As a result, as shown in FIGS. 17A to 17C, it was confirmed that NK cells (NKp46 expression) infiltrated into the brain tissues only in the kainic acid-treatment group.

### Example 9. Confirming that NK Cells have Function to Remove Nerves in Central Nervous System Excitotoxicity Mouse Model

To examine whether NK cells are also able to selectively remove injured or abnormal nerves in the central nerve system, analysis of behavioral responses and measurement of ligand expression of NK cells were performed in central nervous system excitotoxicity-induced mice.

In detail, Nkp46-DTR transgenic mice that conditionally lack endogenous NK cells (normal when treated with PBS, and lack of NK cells when treated with Diphtheria toxin (DTx)) were treated with a high concentration of kainic acid (35 mg/kg) which acts on the central nervous system to cause excitotoxicity and to induce degeneration, and behavioral responses were analyzed based on Racine's stage criteria. Criteria used when seizure behaviors were observed are shown in [Table 1] below, and a higher stage indicates more serious.

TABLE 1

| Stage | Behavior |
|---|---|
| 1 | Mouth and facial movement |
| 2 | Head nodding |
| 3 | Forelimb clonus |
| 4 | Rearing with forelimb clonus |
| 5 | Rearing and falling with forelimb clonus (generalized |
| | motor convulsions) |

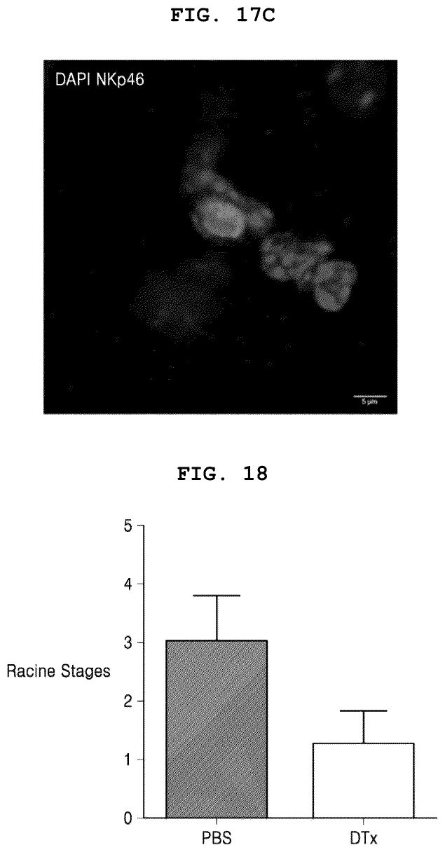

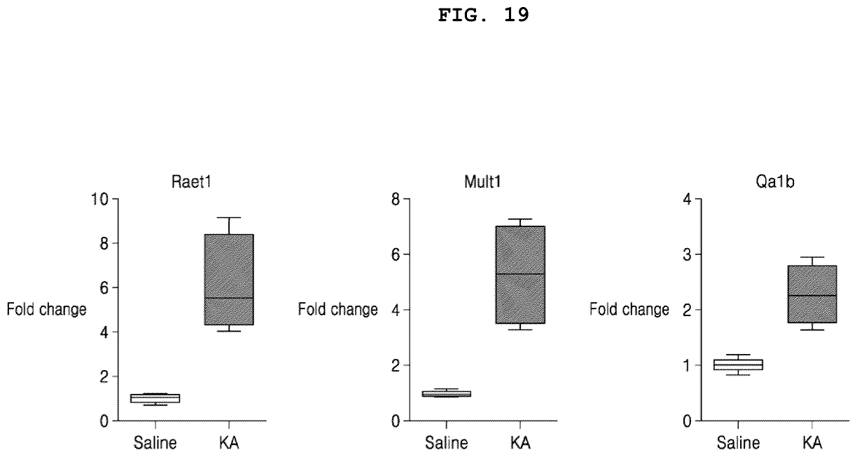

FIG. 18 shows results of analyzing behavioral responses occurring after induction of excitotoxicity and degeneration of the central nervous system in Nkp46-DTR transgenic mice. FIG. 19 shows results of measuring ligand expression of NK cells in hippocampal tissues on day 3 after induction of excitotoxicity and degeneration of the central nervous system.

As a result, as shown in FIG. 18, it was confirmed that the behavioral responses due to neurodegeneration appeared less when NK cells were deficient.

Further, as shown in FIG. 19, increase of Raet1 and Mult1, which are activating ligands of NK cells, was observed, and expression of an inhibitory ligand, Qa1b, was also slightly increased. However, since target cell attack of NK cells is regulated by the relative expression levels of the activating ligands and the inhibitory ligand of the cells, these results mean that the injured nerve-removing function of NK cells is also activated in the central nerve system.

The above results suggest that mouse NK cells are able to degenerate the injured central nerve causing excitotoxicity, which may be mediated by the activating ligands (RAE1 and MULT1) highly expressed in the injured mouse nerve. It means that NK cells may be usefully applied in the treatment of seizures or symptoms caused by nerve degeneration, which occurs due to nervous system diseases caused by nerve injury or abnormal nerves in the central nervous system.

### Example 10. Confirming that Adoptive Transfer of NK Cells has Function to Alleviate Chronic Neuropathic Pain in the Partial Sciatic Nerve Crush Injury Mouse Model

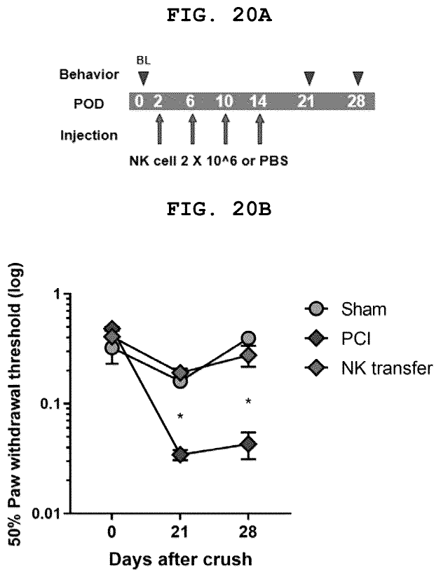

To examine the prophylactic effects of adoptive transfer of mouse NK cells on chronic neuropathic pain in mice, IL-2 activated mouse NK cells (2×10⁶ cells) were intravenously (i.v.) injected into the injured mice on day 2, 6, 10, and 14 after the partial crush injury (PCI), which is an early time point from the injury, and mechanical hypersensitivity was assessed by measuring responses to von Frey hair stimulation of the lateral hind paw (FIG. 20A). The adoptive transfer of the mouse NK cells during the early time point resulted in significantly higher mechanical thresholds in the hind paw of the previously crushed limb, compared to control (PCI) (FIG. 20B). The thresholds of the adoptive transferred group were comparable to those of sham, which indicated the early treatment of the activated mouse NK cells could prevent persistent tactile hypersensitivity in the partial crush injured mouse model.

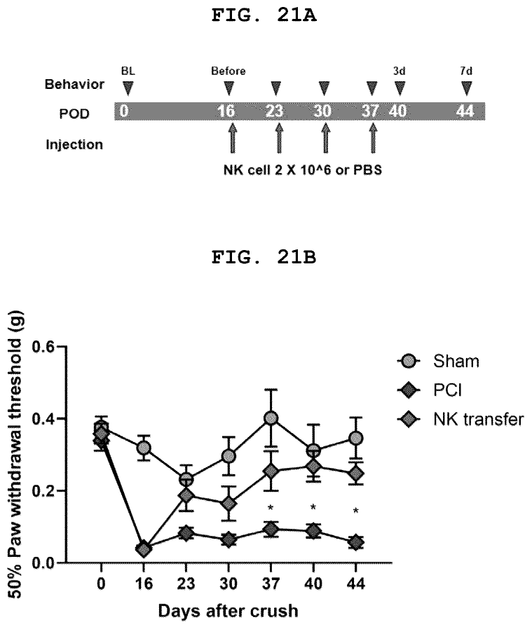

Furthermore, to examine the therapeutic effects of adoptive transfer of mouse NK cells, IL-2 stimulated mouse NK cells (2×10⁶ cells) were i.v. injected to the injured mice on day 16, 23, 30, and 37 after the partial crush injury, and mechanical hypersensitivity was assessed by measuring responses to von Frey hair stimulation of the lateral hind paw (FIG. 21A). The injection of the mouse NK cells after 37 days from the injury resulted in significantly higher mechanical thresholds in the hind paw of the previously crushed limb, compared to control (PCI), which indicated the adoptive transfer of mouse NK cells alleviated persistent tactile hypersensitivity in mice (FIG. 21B).

Taken together, these data suggest that the adoptive transfer of NK cells can serve as prophylactic and therapeutic biological agents for chronic neuropathic pain.

### Example 11. Confirming that Activated Human NK Cells Induce Cytotoxicity in Human iPSC-Derived Sensory Neurons by NKG2D-Mediated Mechanism

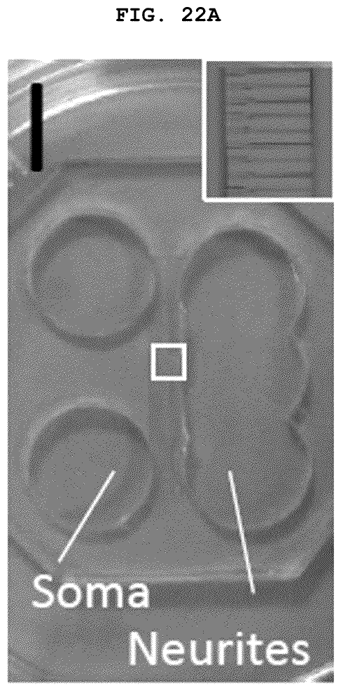

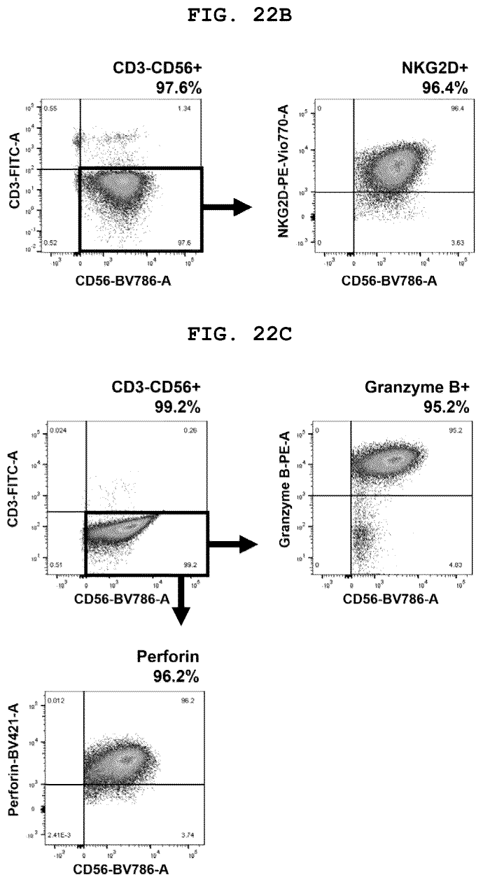

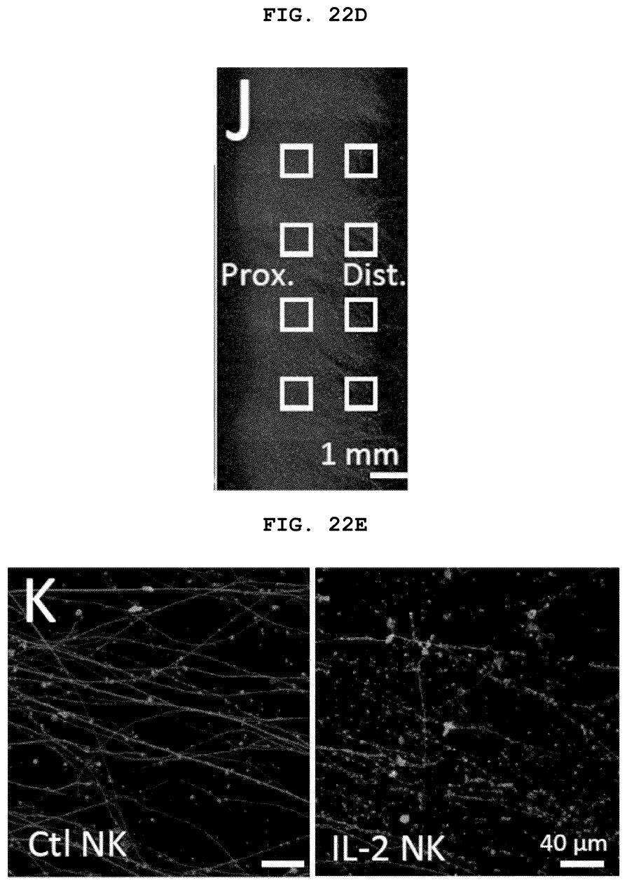

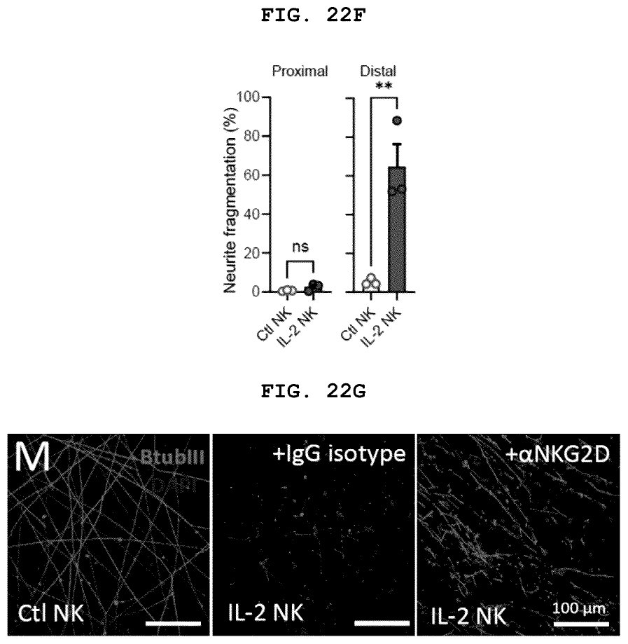

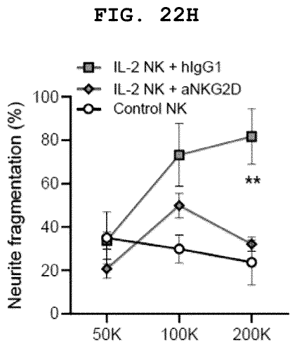

To investigate whether NK cell-mediated cytotoxicity to sensory neurons occurs not only in mice but also in humans, human iPSC (induced pluripotent stem cell) derived sensory neurons were prepared (FIG. 22A) and were co-cultured with pre-activated human NK cells. The activation markers and activation status of the pre-activated human NK cells were assessed by flow cytometry, and the neurite fragmentation was measured in immunofluorescence-stained images. The analysis demonstrated that at least 96% of the pre-activated human NK cells were CD3-CD56⁺NKG2D⁺, at least 96% of the CD3⁻CD56⁺ pre-activated human NK cells expressed perforin, and at least 95% of the CD3⁻CD56⁺ pre-activated human NK cells expressed granzyme B (FIGS. 22B and 22C). The human iPSC-derived sensory neurons were stained against β-tubulin III to visualize neurite compartment (FIG. 22D), and the immunofluorescence-stained images of the co-culture of the iPSC-derived neurons and NK cells were analyzed to assess the level of neurite fragmentation, which indicated NK cell-mediated cytotoxicity (FIGS. 22E and 22F). Co-culturing with IL-2-primed human NK cells resulted in a significant increase of distal neurite fragmentation, compared with co-culture with the un-stimulated NK cells (FIG. 22F).

To determine whether the cytotoxicity was mediated by NKG2D-NKG2D ligand interaction, the human iPSC-derived sensory neurons were co-cultured with unstimulated, or IL-2-primed human NK cells in the absence or presence of anti-NKG2D antibody (Cat. no. 320813, RRID:AB_2561488; Biolegend) that could block the interaction between NKG2D and its ligands. Co-culturing of IL-2-primed NK cells in the absence of anti-NKG2D antibody resulted in highly increased neurite fragmentation. In contrast, addition of anti-NKG2D antibody significantly reduced the neurite fragmentation and the level of neurite fragmentation was comparable to that induced by unstimulated NK cells (FIGS. 22G and 22H). These findings indicated that the NK cell-mediated cytotoxicity occurred through the interaction between NKG2D and its ligands.

The above results suggest that NK cells can induce cytotoxicity and clearance of injured neurons expressing NKG2D ligands through the interaction with NKG2D receptor on NK cells in humans as well as mice.

### Example 12. Human-Derived Activated NK Cells for Killing Injured Peripheral Nerves (Prophetic Example)

Human natural killer (NK) cells are isolated from peripheral blood of a human donor and activated ex vivo by culturing in the presence of interleukin-2 (IL-2) prior to administration.

The activated NK cells are characterized as expressing NKp46 and NKG2D and exhibiting cytotoxic effector functions consistent with granzyme B and perforin expression, as described for activated NK cells herein.

In this prophetic example, the activated human NK cells are administered to a subject having injured or abnormal peripheral sensory nerves. The injured or abnormal nerves exhibit stress-induced expression of NKG2D ligands as a result of nerve damage.

It is expected that the activated human NK cells selectively kill the injured or abnormal peripheral nerves through NKG2D receptor-ligand interactions, while sparing healthy peripheral nerves that do not express such ligands.

### Example 13. Selective Killing of Injured Peripheral Nerves without Damage to Normal or Central Neurons (Prophetic Example)

Activated NK cells prepared as described herein are administered to a subject having injured or abnormal peripheral nerves.

In this prophetic example, injured or abnormal peripheral nerves are expected to exhibit upregulated expression of NKG2D ligands, whereas normal central nervous system neurons are not expected to exhibit NKG2D ligand expression at levels sufficient to trigger NK cell-mediated cytotoxicity under non-pathological conditions.

Accordingly, the activated NK cells preferentially kill injured or abnormal peripheral nerves via NKG2D-dependent mechanisms, while normal peripheral nerves and central nervous system neurons remain substantially unaffected.

Thus, selective killing of injured or abnormal nerves is achieved without inducing widespread neurotoxicity or damage to healthy neural tissue.

### Example 14. Applicability Across Distinct Peripheral Neuropathic Conditions (Prophetic Examples)

Activated NK cells prepared according to the present disclosure are administered to subjects having injured or abnormal peripheral nerves arising from different pathological conditions, including diabetic neuropathy, chemotherapy-induced peripheral neuropathy, viral infection-associated neuropathy, and traumatic nerve injury.

In each condition, injured or abnormal peripheral nerves are expected to exhibit increased expression of stress-induced ligands recognizable by NK cells, including NKG2D ligands.

Accordingly, the activated NK cells are expected to kill injured or abnormal nerves across these distinct neuropathic conditions through a shared biological mechanism, without requiring substantial modification beyond routine, case-specific optimization of the NK cell preparation or administration protocol.

### Example 15. Human-Derived Activated NK Cells for Killing Injured Central Nerves (Prophetic Example)

Activated NK cells prepared as described herein are administered to a subject having injured or abnormal central nerves.

In this prophetic example, the activated human NK cells are administered to a subject having injured or abnormal central nerves. The injured or abnormal nerves exhibit stress-induced expression of NKG2D ligands as a result of nerve damage.

It is expected that the activated human NK cells selectively kill the injured or abnormal central nerves through NKG2D receptor-ligand interactions, while sparing healthy central nerves that do not express such ligands.

### Example 16. Selective Killing of Injured Central Nerves without Damage to Normal Nerves (Prophetic Example)

Activated NK cells prepared as described herein are administered to a subject having injured or abnormal central nerves.

In this prophetic example, injured or abnormal central nerves are expected to exhibit upregulated expression of NKG2D ligands, whereas normal central nerves are not expected to exhibit NKG2D ligand expression at levels sufficient to trigger NK cell-mediated cytotoxicity under non-pathological conditions.

Accordingly, the activated NK cells preferentially kill injured or abnormal central nerves via NKG2D-dependent mechanisms, while normal central nerves remain substantially unaffected.

Thus, selective killing of injured or abnormal nerves is achieved without inducing widespread neurotoxicity or damage to healthy nervous tissue.

### Example 17. Applicability Across Distinct Central Neuropathic Conditions (Prophetic Examples)

Activated NK cells prepared as described herein are administered to subjects having injured or abnormal central nerves associated with different pathological conditions, including epilepsy, multiple sclerosis, amyotrophic lateral sclerosis, Alzheimer's disease, Lewy dementia, Huntington's disease, Parkinson's disease, schizophrenia, traumatic brain injury, stroke, Pick's disease, Creutzfeldt-Jakob disease, progressive supernuclear palsy, multiple system atrophy, corticobasal degeneration, spinocerebellar degeneration, cerebellar atrophy, post-traumatic stress disorder, amnesia, vascular dementia, and cerebral infarction.

In each condition, injured or abnormal central nerves are expected to exhibit increased expression of stress-induced ligands recognizable by NK cells, including NKG2D ligands.

Accordingly, the activated NK cells are expected to kill injured or abnormal nerves across these distinct neuropathic conditions through a shared biological mechanism, without requiring substantial modification beyond routine, case-specific optimization of the NK cell preparation or administration protocol.

1. A method for killing injured or abnormal nerves, comprising contacting the injured or abnormal nerves with isolated natural killer (NK) cells or a cell population thereof,
wherein the injured or abnormal nerves express NKG2D ligands, and the NK cells or the cell population thereof recognize and remove the injured or abnormal nerves via NKG2D receptor-ligand interaction.

2. The method of claim 1, the isolated NK cells or the cell population thereof are activated with IL-2 prior to the contact.

3. The method of claim 2, where the activated NK cells or the cell population thereof are NKG2D⁺.

4. A method for preventing or treating a nervous system disease caused by nerve injury or abnormal nerves, the method comprising:
administering to a subject in need thereof an effective amount of an isolated natural killer (NK) cell, a cell population thereof, a substance increasing NK cell activity, or a combination thereof,
wherein the injured or abnormal nerves express NKG2D ligands and the NK cells or the cell population thereof recognize and remove the injured or abnormal nerves via NKG2D receptor-ligand interaction.

5. The method of claim 4, the isolated NK cell or the cell population thereof was administered to the subject in need thereof via intravenous (i.v.) injection.

6. The method of claim 4, wherein the isolated NK cell or the cell population thereof has increased activity or is genetically modified to have increased activity, as compared with a NK progenitor cell.

7. The method of claim 6, wherein the genetically modified NK cell or cell population thereof is modified to express chimeric antigen receptor (CAR) or homing receptor.

8. The method of claim 7, wherein the chimeric antigen receptor comprises an extracellular domain, a transmembrane domain, and an intracellular stimulatory domain.

9. The method of claim 4, wherein the substance increasing NK cell activity is one or more selected from the group consisting of interferon, interleukin, an interleukin-antibody complex, an agonist of NK cell activating receptor, an antibody against immunoglobulin-like receptor, an antibody against glucocorticoid-induced TNF-related protein (GITR), an antibody against CD137, an antibody against CD27, an antibody against OX40, an antibody against CTLA-4, an antibody against PD-1, and an antibody against NKG2A.

10. The method of claim 9, wherein the interleukin is any one selected from the group consisting of IL-2, IL-5, IL-8, IL-12, IL-15, IL-18, IL-21, and a combination thereof.

11. The method of claim 9, wherein the agonist of NK cell activating receptor is bispecific killer engagers (BiKEs) or trispecific killer engagers (TrikEs).

12. The method of claim 4, wherein the nerve injury is neuropraxia, axonotmesis, or neurotmesis.

13. The method of claim 4, wherein the nervous system disease caused by nerve injury or abnormal nerves is a nervous system disease caused by nerve injury or abnormal nerves of the central nervous system or a nervous system disease caused by nerve injury or abnormal nerves of the peripheral nervous system.

14. The method of claim 13, wherein the nervous system disease caused by nerve injury or abnormal nerves of the central nervous system is any one selected from the group consisting of organic diseases and dysfunctions of the central nervous system, epilepsy, multiple sclerosis, amyotrophic lateral sclerosis, Alzheimer's disease, Lewy dementia, Huntington's disease, Parkinson's disease, schizophrenia, traumatic brain injury, stroke, Pick's disease, Creutzfeldt-Jakob disease, progressive supernuclear palsy, multiple system atrophy, corticobasal degeneration, spinocerebellar degeneration, cerebellar atrophy, post-traumatic stress disorder, amnesia, vascular dementia, and cerebral infarction.

15. The method of claim 13, wherein the nervous system disease caused by nerve injury or abnormal nerves of the peripheral nervous system is any one selected from the group consisting of peripheral neuropathy, diabetic neuropathy, peripheral neuropathic pain, chemotherapy-induced peripheral neuropathy, complex regional pain syndrome, optic neuropathy, mononeuropathy, mononeuropathy multiplex (mononeuritis multiplex), polyneuropathy, Guillain-Barre syndrome (acute inflammatory demyelinating polyneuropathy), chronic inflammatory demyelinating polyneuropathy, hereditary neuropathy, plexus disorder, glaucoma, macular degeneration, amyotrophic lateral sclerosis, progressive muscle atrophy, progressive bulbar palsy, polio, post-polio syndrome, stiff-man syndrome, Isaac's syndrome, myasthenia gravis, neonatal myasthenia, botulism, Lambert-Eaton Myasthenic Syndrome, thoracic outlet syndrome, Charcot-Marie-Tooth disease, fibromyalgia syndrome, and spinal muscular atrophy.

16. The method of claim 15, wherein the peripheral neuropathic pain is any one selected from the group consisting of trigeminal neuralgia, diabetic neuropathic pain, phantom limb pain, viral infection, pain from trauma, pain following chemotherapy, atypical facial pain, and post herpetic neuralgia.

17. The method of claim 15, wherein the diabetic neuropathy is polyneuropathy or focal neuropathy.

18. The method of claim 17, wherein the polyneuropathy is any one selected from the group consisting of hyperglycemic neuropathy, distal symmetric polyneuropathy, autonomic neuropathy, acute sensory neuropathy, acute painful sensory neuropathy, and chronic sensorimotor neuropathy.

19. The method of claim 17, wherein the focal neuropathy is any one selected from the group consisting of cranial neuropathy, truncal neuropathy, limb neuropathy, thoracolumbar radiculoneuropathy, and lumbosacral radiculoplexus neuropathy.

20. The method of claim 4, wherein the isolated NK cell, the cell population thereof, the substance increasing NK cell activity, or the combination thereof removes injured or abnormal nerves.
